SEQUENCE LISTING (22 sequences)

SEQ ID NO: 1 - DNA, 13 bp, Artificial Sequence
  Oligonucleotide used for a probe
cgcaatntaacgc

SEQ ID NO: 2 - DNA, 13 bp, Artificial Sequence
  Oligonucleotide used for a target
gcgttaaattgcg

SEQ ID NO: 3 - DNA, 13 bp, Artificial Sequence
  Oligonucleotide used for a target
gcgttatattgcg

SEQ ID NO: 4 - DNA, 13 bp, Artificial Sequence
  Oligonucleotide used for a target
gcgttagattgcg

SEQ ID NO: 5 - DNA, 13 bp, Artificial Sequence
  Oligonucleotide used for a target
gcgttacattgcg

SEQ ID NO: 6 - DNA, 13 bp, Artificial Sequence
  Oligonucleotide used for a target
gcguuaaauugcg

SEQ ID NO: 7 - DNA, 13 bp, Artificial Sequence
  Oligonucleotide used for a target
gcguuauauugcg

SEQ ID NO: 8 - DNA, 13 bp, Artificial Sequence
  Oligonucleotide used for a target
gcguuagauugcg

SEQ ID NO: 9 - DNA, 13 bp, Artificial Sequence
  Oligonucleotide used for a target
gcguuacauugcg

SEQ ID NO: 10 - DNA, 40 bp, Artificial Sequence
  Oligonucleotide used for a primer
agattaatgtaaaagtgatgtgttgattttatgcatgcca

SEQ ID NO: 11 - DNA, 32 bp, Artificial Sequence
  Oligonucleotide used for a primer
gggcttggtcaatatagaattttggatttccc

SEQ ID NO: 12 - DNA, 20 bp, Artificial Sequence
  Oligonucleotide used for a primer
gtgatctgctccattatttt

SEQ ID NO: 13 - DNA, 21 bp, Artificial Sequence
  Oligonucleotide used for a probe
ctggatncagggggtgcttac

SEQ ID NO: 14 - DNA, 18 bp, Artificial Sequence
  Oligonucleotide used for a primer
ctccatcaagctntcgct

SEQ ID NO: 15 - DNA, 25 bp, Artificial Sequence
  Oligonucleotide used for a primer
cgaaagtagtaatactttagaggcg

SEQ ID NO: 16 - DNA, 26 bp, Artificial Sequence
  Oligonucleotide used for a primer
gaagaagactatgctcgnagatcaga

SEQ ID NO: 17 - DNA, 28 bp, Artificial Sequence
  Oligonucleotide used for a primer
catcagtattgtagaagtaccacatacc

SEQ ID NO: 18 - DNA, 13 bp, Artificial Sequence
  Oligonucleotide used for a primer
cgcaacncaacgc

SEQ ID NO: 19 - DNA, 13 bp, Artificial Sequence
  Oligonucleotide used for a target
gcgttgagttgcg

SEQ ID NO: 20 - DNA, 13 bp, Artificial Sequence
  Oligonucleotide used for a target
gcgttgtgttgcg

SEQ ID NO: 21 - DNA, 13 bp, Artificial Sequence
  Oligonucleotide used for a target
gcgttgggttgcg

SEQ ID NO: 22 - DNA, 13 bp, Artificial Sequence
  Oligonucleotide used for a target
gcgttgcgttgcg

(19) United States
(12) Patent Application Publication
(10) Pub. No.: US 2006/0142311 A1
(43) Pub. Date: Jun. 29, 2006

(54) Prodan-containing nucleotide and use thereof

(75) Inventors: Akimitsu Okamoto, Kyoto-shi (JP); Kazuki Tainaka, Kyoto-shi (JP); Isao Saito, Chiyoda-ku (JP); Norihiko Takahashi, Tokushima-shi (JP)

Correspondence Address: SUGHRUE MION, PLLC, 2100 PENNSYLVANIA AVENUE, N.W., SUITE 800, WASHINGTON, DC, 20037, US

(73) Assignees: KYOTO UNIVERSITY; NIHON UNIVERSITY; OTSUKA PHARMACEUTICAL CO., LTD.

(21) Appl. No.: 11/135,368

(22) Filed: May 24, 2005

(30) Foreign Application Priority Data
Nov. 18, 2004 (JP) 2004-335057

(51) Int. Cl. A61K 31/522; A61K 31/513

(52) U.S. Cl. 514/263.36; 544/276; 514/269; 544/314

(57) ABSTRACT

A compound represented by formula (1): 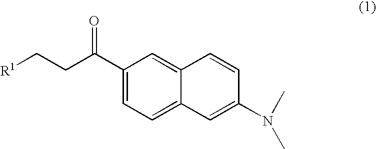
wherein R¹ is a substituent represented by formula (2): 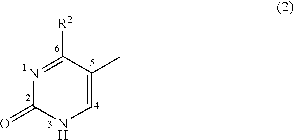
wherein R² is ═O or —NH₂, with the proviso that when R² is ═O, H is attached to the 1-position N of the pyrimidine ring, and the bond between the 1-position N and the 6-position C is a single bond; or a substituent represented by formula (3): 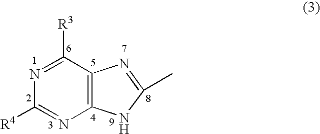
wherein R³ is —OH, ═O, or —NH₂, with the proviso that when R³ is —OH or —NH₂, R⁴ is H; when R³ is ═O, R⁴ is —NH₂; and when R³ is ═O, H is attached to the 1-position N of the purine ring, and the bond between the 1-position N and the 6-position C is a single bond.

### BACKGROUND OF THE INVENTION

(1) Field of the Invention

The present invention relates to a polynucleotide derivative used for the determination of the kind of nucleotide at a particular site in a nucleotide sequence, an intermediate thereof, a probe, a primer, a nucleotide identification reagent, a nucleic acid quantitation reagent, a DNA chip, a method of identifying nucleotides, and a method of quantitating nucleic acids.

(2) Description of the Related Art

Nucleic acid base determination using hybridization has so far been conducted by hybridizing a polynucleotide or an oligonucleotide as a probe with a fluorescently labeled target nucleic acid and measuring the melting temperature. More specifically, bases are identified by a small difference in the melting temperature of a double strand, depending on whether a base at a particular position in the probe pairs with the corresponding base in the target nucleic acid.

In the above method, however, it is necessary to set conditions for each target nucleic acid in order to maximize the melting temperature difference between the case where a base pair is formed and the case where it is not formed. Furthermore, the occurrence of nonspecific adsorption between bases, instability of base pairing, etc. tend to cause detection errors. Such detection errors are further increased when the amount of label of the target nucleic acid must be reduced, when fluorescence is diminished by prolonged operation, and the like.

The use of fluorescently labeled probes to detect target nucleic acid sequences has been proposed in, for example, Japanese Examined Patent Publication No. 1991-71437 (column 4, formula (I); and column 5, lines 5-9), Japanese Unexamined Patent Publication No. 1997-505556 (claim 1; and page 15, lines 11-19), Japanese Unexamined Patent Publication No. 1994-135991 (claim 1; paragraph 0004; and paragraph 0005), and Japanese Unexamined Patent Publication No. 1987-059293 (claim 1; and page 2, lower right column, lines 5-8). In this method, however, it is necessary to include a step of separating hybridized double strands from unreacted fluorescent nucleic acid probes by operations such as washing, etc. When washing is insufficient, nonspecific fluorescence may remain and show false positive results. These fluorescent probes always emit fluorescence and only label target nucleic acids by forming base pairs with the target nucleic acids; they cannot identify particular bases in the target nucleic acids.

Japanese Unexamined Patent Publication No. 2004-166522 (claims, etc.) and Japanese Unexamined Patent Publication No. 2004-168672 (claims, etc.) disclose a method of identifying a particular base in a target nucleic acid, comprising using a polynucleotide derivative wherein one or more nucleotides are substituted by a nucleotide derivative as a probe and hybridizing the polynucleotide derivative with the target nucleic acid. This method identifies a particular base in a target nucleic acid based on the principle that the polynucleotide derivative, when hybridized with the target nucleic acid, has its fluorescence enhanced or quenched, or has its emission wavelength shifted, depending on the kind of nucleotide in the target nucleic acid that pairs with the nucleotide derivative. However, the fluorescence emission wavelength of the polynucleotide derivatives of these patent documents is about 380 to about 420 nm, and fluorescence spectrum measurement needs a special instrument to measure the fluorescence in the short wavelength region near the ultraviolet range.

### BRIEF SUMMARY OF THE INVENTION

Objects of the present invention are to provide a polynucleotide derivative that can be used to identify nucleic acid bases simply and accurately; a nucleoside derivative, a nucleotide derivative and a base compound that can be used as intermediates of the polynucleotide derivative; a probe; a primer; a nucleotide identification reagent; a nucleic acid quantitation reagent; a DNA chip; a method of identifying nucleotides in a target nucleic acid; and a method of quantitating nucleic acids.

In order to achieve the above objects, the present inventors conducted extensive research and obtained the following findings.

(i) A polynucleotide derivative wherein one or more nucleotides are substituted by a nucleotide derivative represented by formula (20): 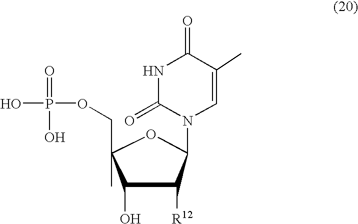
wherein R¹² is —H or —OH. This polynucleotide derivative emits fluorescence. In hybridization with a target nucleic acid, this polynucleotide derivative as a probe has about 4 times as great fluorescence intensity at 490 to 550 nm when the base in the nucleotide in the complementary strand that pairs with the nucleotide derivative is adenine, as when it is guanine, cytosine, thymine, or uracil. The use of this polynucleotide derivative enables the easy and accurate identification of a base at a particular position in the target nucleic acid.

(ii) A polynucleotide derivative wherein one or more nucleotides are substituted by a nucleotide derivative represented by formula (22): 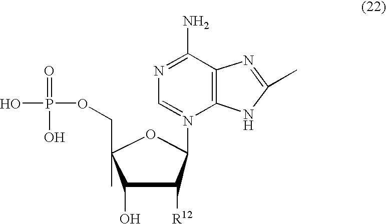
wherein R¹² is —H or —OH. This polynucleotide derivative emits fluorescence. In hybridization with a target nucleic acid, this polynucleotide derivative as a probe has about 3 times as great fluorescence intensity at 490 to 550 nm when the base in the nucleotide in the complementary strand that pairs with the nucleotide derivative is thymine, as when it is adenine, guanine, cytosine, or uracil. The use of this polynucleotide derivative enables the easy and accurate identification of a base at a particular position in the target nucleic acid.

(iii) Similar results can be obtained by the use of a polynucleotide derivative wherein one or more nucleotides are substituted by a nucleotide derivative represented by formula (21): 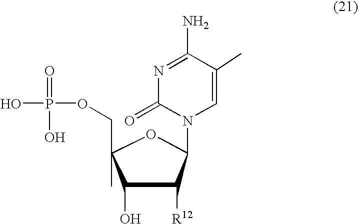
wherein R¹² is —H or —OH, formula (23): 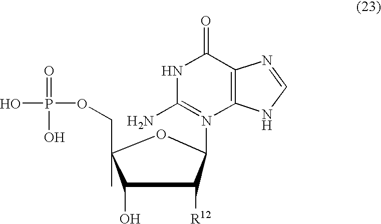
wherein R¹² is —H or —OH, or formula (24): 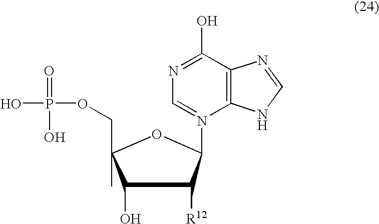
wherein R¹² is —H or —OH.

Based on the above findings, the present invention was accomplished. The invention provides the following compounds, nucleoside or nucleotide derivatives, etc.

1. A compound represented by formula (1): 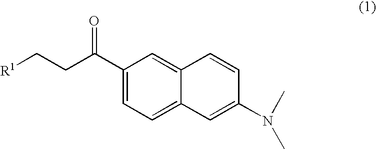
wherein R¹ is a substituent represented by formula (2): 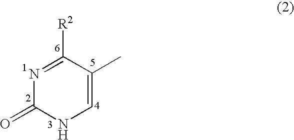
wherein R² is ═O or —NH₂, with the proviso that when R² is ═O, H is attached to the 1-position N of the pyrimidine ring, and the bond between the 1-position N and the 6-position C is a single bond; or a substituent represented by formula (3): 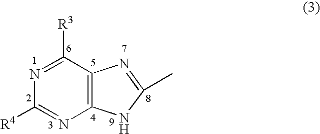
wherein R³ is —OH, ═O, or —NH₂, with the proviso that when R³ is —OH or —NH₂, R⁴ is H; when R³ is ═O, R⁴ is —NH₂; and when R³ is ═O, H is attached to the 1-position N of the purine ring, and the bond between the 1-position N and the 6-position C is a single bond.

2. The compound according to item 1 represented by formula (4):

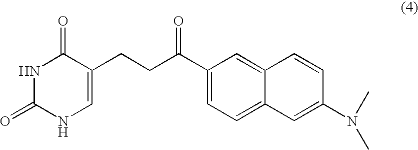

3. The compound according to item 1 represented by formula (5):

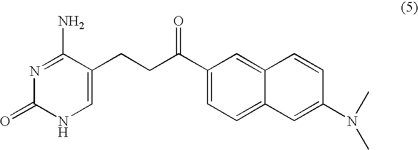

4. The compound according to item 1 represented by formula (6):

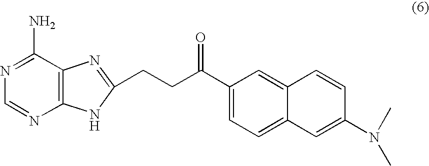

5. The compound according to item 1 represented by formula (7):

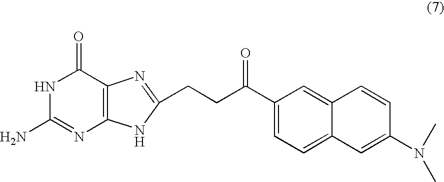

6. The compound according to item 1 represented by formula (8):

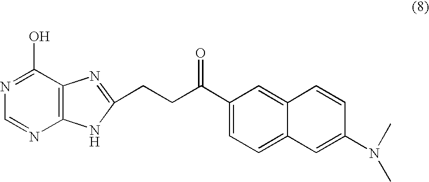

7. A nucleoside or nucleotide derivative represented by formula (9): 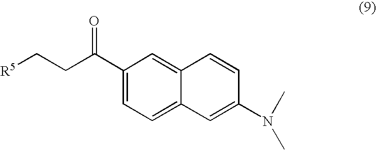
wherein R⁵ is a substituent represented by formula (10): 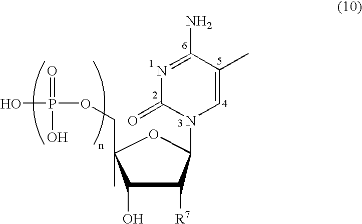
wherein R⁷ is —H or —OH; n is 0, 1, 2, or 3; and R⁶ is ═O or —NH₂, with the proviso that when R⁶ is ═O, H is attached to the 1-position N of the pyrimidine ring, and the bond between the 1-position N and the 6-position C is a single bond; or a substituent represented by formula (11): 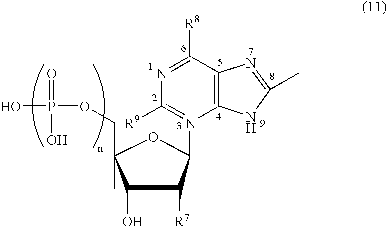
wherein R⁷ is —H or —OH; n is 0, 1, 2, or 3; and R⁸ is —OH, ═O, or —NH₂, with the proviso that when R⁸ is —OH or —NH₂, R⁹ is H; when R⁸ is ═O, R⁹ is —NH₂; and when R⁸ is ═O, H is attached to the 1-position N of the purine ring, and the bond between the 1-position N and the 6-position C is a single bond. 8. A nucleoside or nucleotide derivative according to item 7 represented by formula (12): 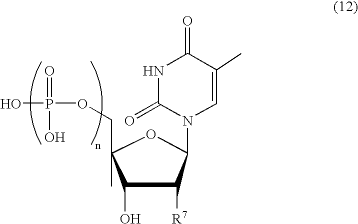
wherein R⁷ is —H or —OH; and n is 0, 1, 2, or 3.

9. A nucleoside or nucleotide derivative according to item 7 represented by formula (13): 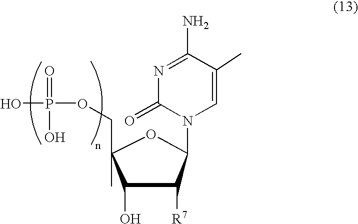
wherein R⁷ is —H or —OH; and n is 0, 1, 2, or 3.

10. A nucleoside or nucleotide derivative according to item 7 represented by formula (14): 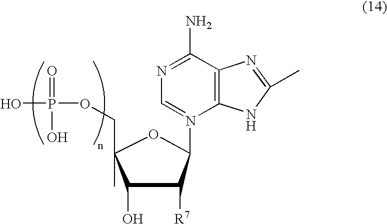
wherein R⁷ is —H or —OH; and n is 0, 1, 2, or 3.

11. A nucleoside or nucleotide derivative according to item 7 represented by formula (15): 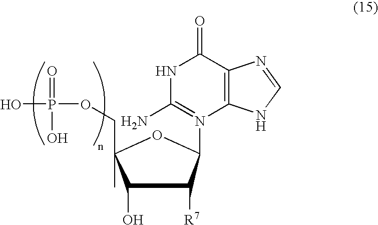
wherein R⁷ is —H or —OH; and n is 0, 1, 2, or 3.

12. A nucleoside or nucleotide derivative according to item 7 represented by formula (16): 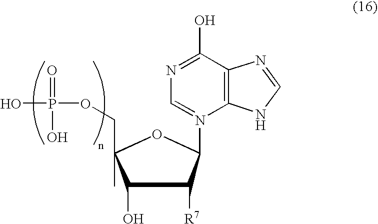
wherein R⁷ is —H or —OH; and n is 0, 1, 2, or 3.

13. A polynucleotide derivative wherein one or more nucleotides are substituted by a nucleotide derivative represented by formula (17): 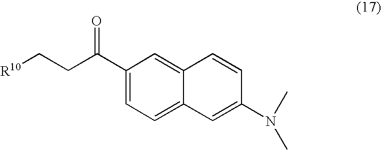
wherein R¹⁰ is a substituent represented by formula (18): 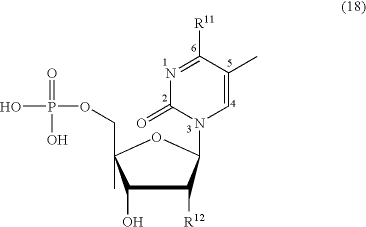
wherein R¹² is —H or —OH; and R¹¹ is ═O or —NH₂, with the proviso that when R¹¹ is ═O, H is attached to the 1-position N of the pyrimidine ring, and the bond between the 1-position N and the 6-position C is a single bond; or a substituent represented by formula (19): 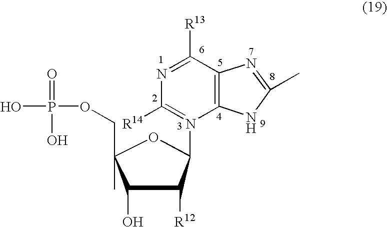
wherein R¹² is —H or —OH; and R¹³ is —OH, ═O, or —NH₂, with the proviso that when R¹³ is —OH or —NH₂, R¹⁴ is H; when R¹³ is ═O, R¹⁴ is —NH₂; and when R¹³ is ═O, H is attached to the 1-position N of the purine ring, and the bond between the 1-position N and the 6-position C is a single bond.

14. A polynucleotide derivative according to item 13 wherein one or more nucleotides are substituted by a nucleotide derivative represented by formula (20): 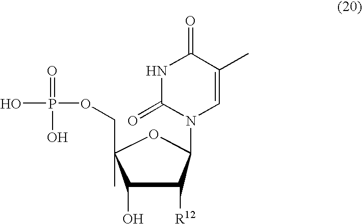
wherein R¹² is —H or —OH.

15. A polynucleotide derivative according to item 13 wherein one or more nucleotides are substituted by a nucleotide derivative represented by formula (21): 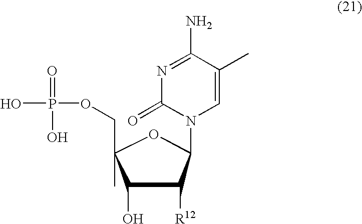
wherein R¹² is —H or —OH.

16. A polynucleotide derivative according to item 13 wherein one or more nucleotides are substituted by a nucleotide derivative represented by formula (22): 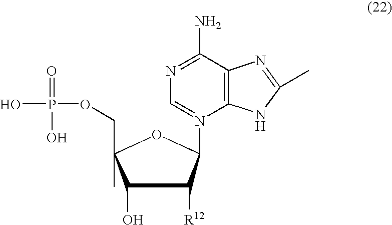
wherein R¹² is —H or —OH.

17. A polynucleotide derivative according to item 13 wherein one or more nucleotides are substituted by a nucleotide derivative represented by formula (23): 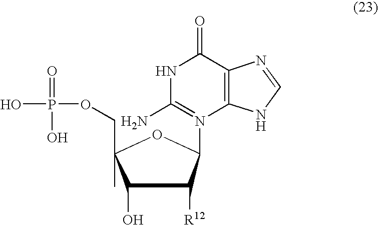
wherein R¹² is —H or —OH.

18. A polynucleotide derivative according to item 13 wherein one or more nucleotides are substituted by a nucleotide derivative represented by formula (24): 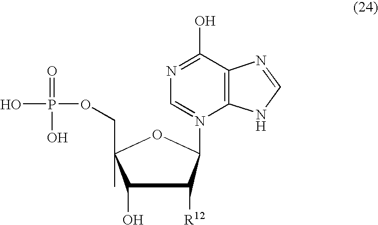
wherein R₁₂ is —H or —OH.

19. A compound represented by formula (25): 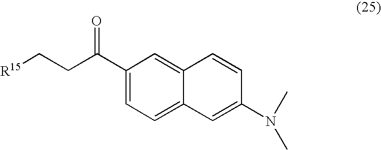
wherein R¹⁵ is a substituent represented by formula (26): 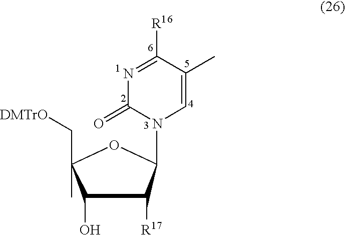
wherein R¹⁷ is —H or —OH; DMTrO— is a dimethoxytrityl group; and R¹⁶ is ═O or —NH₂, with the proviso that when R¹⁶ is ═O, H is attached to the 1-position N of the pyrimidine ring, and the bond between the 1-position N and the 6-position C is a single bond; or a substituent represented by formula (27): 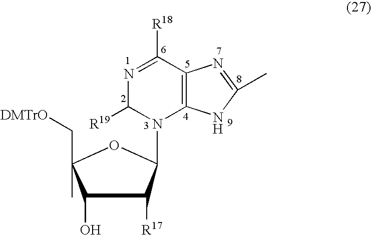
wherein R¹⁷ is —H or —OH; DMTrO— is a dimethoxytrityl group; and R¹⁸ is —OH, ═O, or —NH₂, with the proviso that when R¹⁸ is —OH or —NH₂, R¹⁹ is H; when R¹⁸ is ═O, R¹⁹ is —NH₂; and when R¹⁸ is ═O, H is attached to the 1-position N of the purine ring, and the bond between the 1-position N and the 6-position C is a single bond.

20. A compound according to item 19 represented by formula (28): 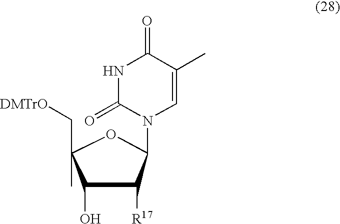
wherein R¹⁷ is —H or —OH; and DMTrO— is a dimethoxytrityl group.

21. A compound according to item 19 represented by formula (29): 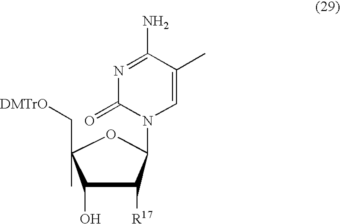
wherein R¹⁷ is —H or —OH; and DMTrO— is a dimethoxytrityl group.

22. A compound according to item 19 represented by formula (30): 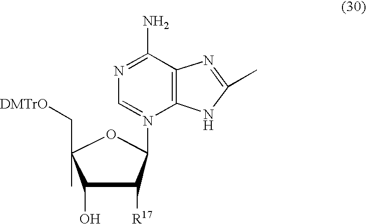
wherein R¹⁷ is —H or —OH; and DMTrO— is a dimethoxytrityl group.

23. A compound according to item 19 represented by formula (31): 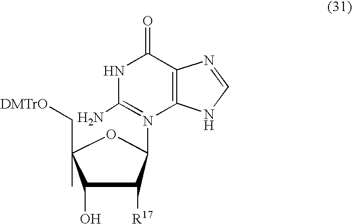
wherein R¹⁷ is —H or —OH; and DMTrO— is a dimethoxytrityl group.

24. A compound according to item 19 represented by formula (32): 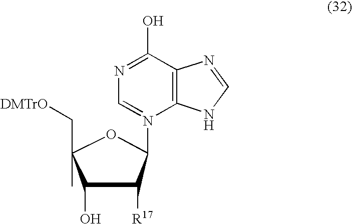
wherein R¹⁷ is —H or —OH; and DMTrO— is a dimethoxytrityl group.

25. A compound represented by formula (33): 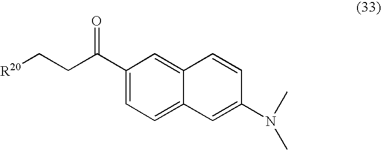
wherein R²⁰ is a substituent represented by formula (34): 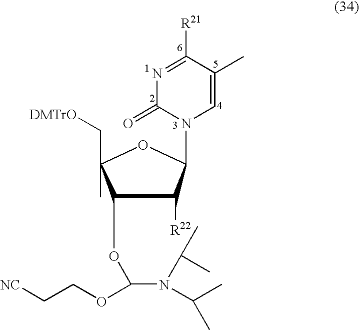
wherein R²² is —H or —OH; DMTrO— is a dimethoxytrityl group; and R²¹ is ═O or —NH₂, with the proviso that when R²¹ is ═O, H is attached to the 1-position N of the pyrimidine ring, and the bond between the 1-position N and the 6-position C is a single bond; or a substituent represented by formula (35): 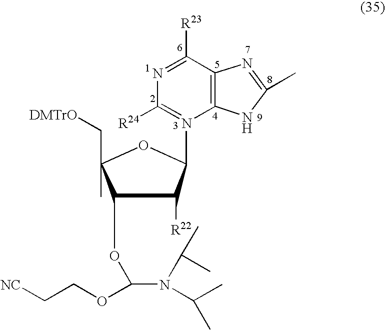
wherein R²² is —H or —OH; DMTrO— is a dimethoxytrityl group; and R²³ is —OH, ═O, or —NH₂, with the proviso that when R²³ is —OH or —NH₂, R²⁴ is H; when R²³ is ═O, R²⁴ is —NH₂; and when R²³ is ═O, H is attached to the 1-position N of the purine ring, and the bond between the 1-position N and the 6-position C is a single bond.

26. A compound according to item 25 represented by formula (36): 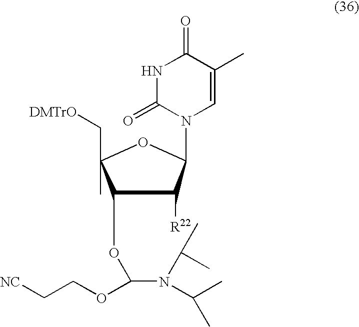
wherein R¹⁷ is —H or —OH, and DMTrO— is a dimethoxytrityl group.

27. A compound according to item 25 represented by formula (37): 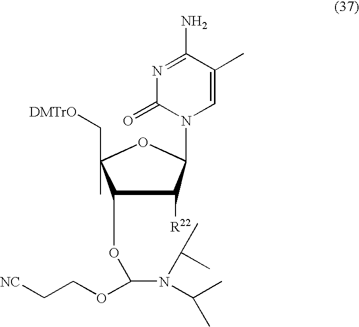
wherein R¹⁷ is —H or —OH; and DMTrO— is a dimethoxytrityl group.

28. A compound according to item 25 represented by formula (38): 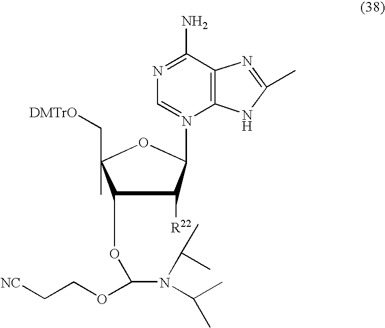
wherein R¹⁷ is —H or —OH; and DMTrO— is a dimethoxytrityl group.

29. A compound according to item 25 represented by formula (39): 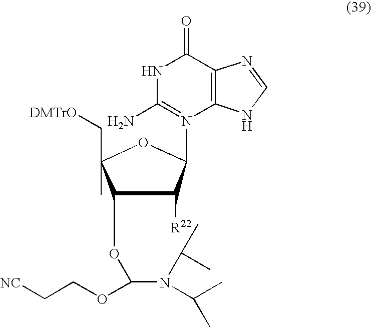
wherein R¹⁷ is —H or —OH; and DMTrO— is a dimethoxytrityl group.

30. A compound according to item 25 represented by formula (40): 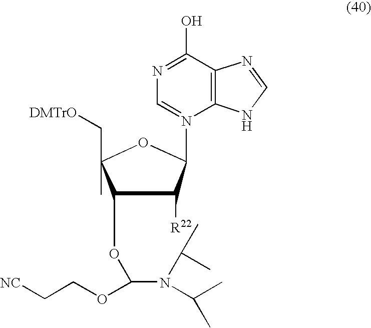
wherein R¹⁷ is —H or —OH; and DMTrO— is a dimethoxytrityl group.

31. A probe comprising a polynucleotide derivative according to any one of items 13 to 18.

32. A nucleotide identification reagent comprising a probe according to item 31.

33. A nucleic acid quantitation reagent comprising a probe according to item 31.

34. A DNA chip wherein one or more polynucleotide derivatives according to any one of items 13 to 18 are immobilized or adsorbed onto a substrate.

35. A method of identifying a nucleotide in a target nucleic acid, comprising the steps of:

hybridizing a polynucleotide derivative according to any one of items 13 to 18 with a target nucleic acid in a sample;

measuring a fluorescence spectrum of the hybridization product; and

comparing the fluorescence spectrum of the hybridization product with a fluorescence spectrum of the polynucleotide derivative before hybridization, thereby identifying a nucleotide at a particular position in the target nucleic acid.

36. A primer comprising a polynucleotide derivative according to any one of items 13 to 18.

37. A nucleic acid quantitation reagent comprising a primer according to item 36.

38. A method of quantitating a target nucleic acid in a sample, comprising the steps of:

hybridizing a polynucleotide derivative according to any one of items 13 to 18 with a target nucleic acid in a sample;

measuring the fluorescence intensity of the hybridization product; and

comparing the fluorescence intensity of the hybridization product with the fluorescence intensity of a hybridization product obtained by hybridizing the above polynucleotide derivative with a target nucleic acid whose concentration is known, thereby determining the concentration of the target nucleic acid.

The present invention provides a polynucleotide derivative that can be used to identify a nucleic acid base easily and accurately; and a nucleoside derivative, a nucleotide derivative and a base compound that can be used as intermediates of the polynucleotide derivative.

Furthermore, in hybridization with a target nucleic acid, the polynucleotide derivative of the invention, which has one or more nucleotide derivatives of the invention, provides much greater fluorescence intensity when the base in the nucleotide in the target nucleic acid that pairs with the nucleotide derivative is a particular base than when it is any other base. The base in the target nucleic acid can be thereby identified.

The fluorescence emission of the polynucleotide derivative of the invention combined with the target nucleic acid may be measured by standard methods over a wavelength range of about 300 to about 650 nm, and particularly about 450 to about 650 nm. The emission spectrum usually has a peak at about 490 to about 550 nm. Therefore, it is not necessary to use a special instrument that detects ultraviolet light.

### BRIEF DESCRIPTION OF THE DRAWING

FIG. 1 shows a synthetic procedure for a sugar derivative used in the synthesis of the nucleotide or nucleoside derivative of the present invention.

FIG. 2 shows a synthetic procedure for an amidite as a synthetic intermediate of the oligonucleotide derivative of the present invention.

FIG. 3 shows the mass spectrum of the oligonucleotide derivative of Example 3.

FIG. 4 shows the HPLC pattern of the enzymatic degradation product of the oligonucleotide derivative of Example 3.

FIG. 5 shows the change of fluorescence spectra caused by the hybridization of an oligodeoxyribonucleotide derivative with various DNAs.

FIG. 6 shows the change of fluorescence spectra caused by the hybridization of an oligodeoxyribonucleotide derivative with various RNAs.

FIG. 7 shows gene polymorphism detection and nucleic acid quantitation by real-time SSDA using an oligodeoxynucleotide derivative as a probe.

FIG. 8 shows gene polymorphism detection and nucleic acid quantitation by real-time PCR using an oligodeoxynucleotide derivative as a probe.

FIG. 9 shows nucleic acid quantitation by real-time PCR using an oligodeoxynucleotide derivative as a primer.

FIG. 10 shows a synthetic procedure for an amidite as a synthetic intermediate of the oligonucleotide derivative of the present invention.

FIG. 11 shows the HPLC pattern of the enzymatic degradation product of the oligonucleotide derivative of Example 10.

FIG. 12 shows the change of fluorescence spectra caused by the hybridization of an oligodeoxyribonucleotide derivative with various DNAs.

### DETAILED DESCRIPTION OF THE INVENTION

The present invention is described below in detail.

(I) Compound of the Present Invention

<Base Compound>

The compound of the present invention is a novel compound represented by formula (1): 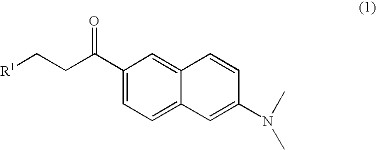
wherein R¹ is a substituent represented by formula (2): 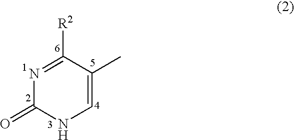
wherein R² is ═O or —NH₂, with the proviso that when R² is ═O, H is attached to the 1-position N of the pyrimidine ring, and the bond between the 1-position N and the 6-position C is a single bond; or a substituent represented by formula (3): 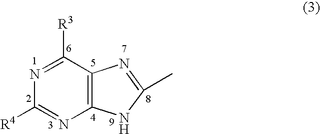
wherein R³ is —OH, ═O, or —NH₂, with the proviso that when R³ is —OH or —NH₂, R⁴ is H; when R³ is ═O, R⁴ is —NH₂; and when R³ is ═O, H is attached to the 1-position N of the purine ring, and the bond between the 1-position N and the 6-position C is a single bond.

Examples of compounds represented by formula (1) include the following five compounds. The compound of formula (4) is a uracil derivative; the compound of formula (5) is a cytosine derivative; the compound of formula (6) is an adenine derivative; the compound of formula (7) is a guanine derivative; and the compound of formula (8) is a hypoxanthine derivative. 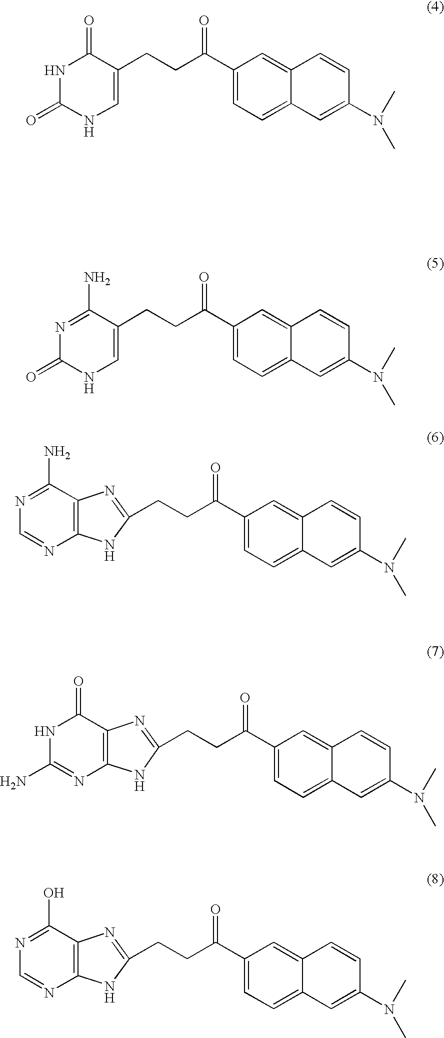
<Nucleoside Derivative/Nucleotide Derivative>

The nucleoside derivative or nucleotide derivative of the present invention is a novel compound represented by formula (9): 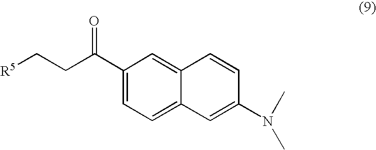
wherein R⁵ is a substituent represented by formula (10): 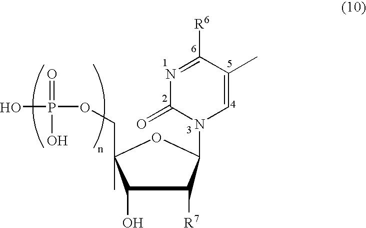
wherein R⁷ is —H or —OH; n is 0, 1, 2, or 3; and R⁶ is ═O or —NH₂, with the proviso that when R⁶ is ═O, H is attached to the 1-position N of the pyrimidine ring, and the bond between the 1-position N and the 6-position C is a single bond; or a substituent represented by formula (11): 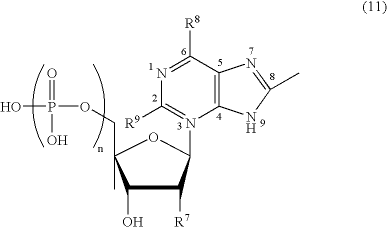
wherein R⁷ is —H or —OH; n is 0, 1, 2, or 3; and R⁸ is —OH, ═O, or —NH₂, with the proviso that when R⁸ is —OH or —NH₂, R⁹ is H; when R⁸ is ═O, R⁹ is —NH₂; and when R⁸ is ═O, H is attached to the 1-position N of the purine ring, and the bond between the 1-position N and the 6-position C is a single bond.

Examples of compounds represented by formula (9) include the nucleoside or nucleotide derivatives represented by formulae (12) to (16). In formulae (12) to (16), R⁷ is —H or —OH; and n is 0, 1, 2, or 3.

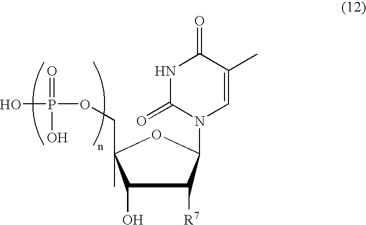

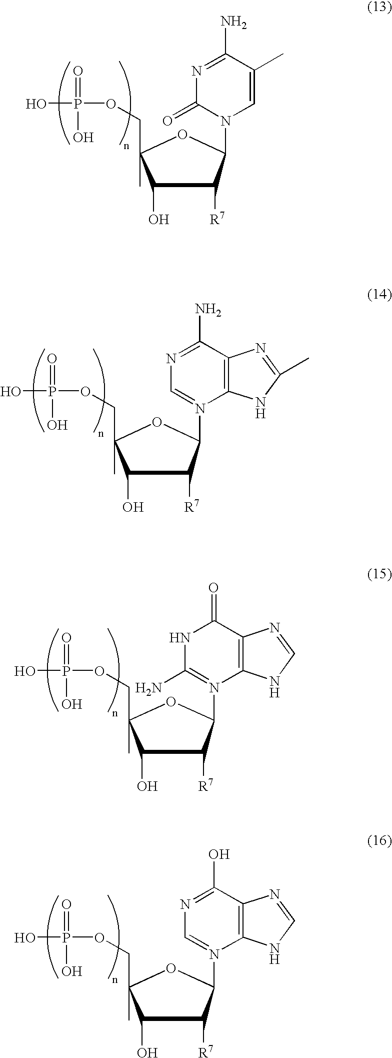

When R⁷ is a hydrogen atom, the above compounds are deoxyribonucleoside derivatives or deoxyribonucleotide derivatives. When R⁷ is a hydroxyl group, the above compounds are ribonucleoside derivatives or ribonucleotide derivatives.

When n is 0, i.e., when no phosphate group is present, the above compounds are nucleoside derivatives. When n is 1, 2, or 3, the above compounds are nucleotide derivatives.

<Polynucleotide Derivative>

The polynucleotide derivative of the present invention is a polynucleotide derivative wherein one or more nucleotides are substituted by a nucleotide derivative represented by formula (17): 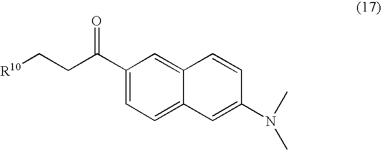
wherein R¹⁰ is a substituent represented by formula (18): 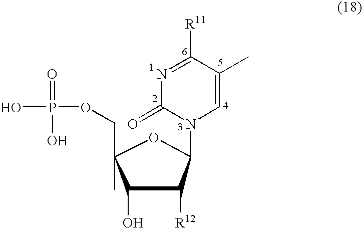
wherein R¹² is —H or —OH; and R¹¹ is ═O or —NH₂, with the proviso that when R¹¹ is ═O, H is attached to the 1-position N of the pyrimidine ring, and the bond between the 1-position N and the 6-position C is a single bond; or a substituent represented by formula (19): 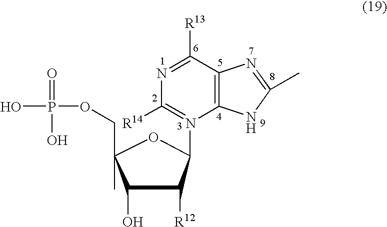
wherein R¹² is —H or —OH; and R¹³ is —OH, ═O, or —NH₂, with the proviso that when R¹³ is —OH or —NH₂, R¹⁴ is H; when R¹³ is ═O, R¹⁴ is —NH₂; and when R¹³ is ═O, H is attached to the 1-position N of the purine ring, and the bond between the 1-position N and the 6-position C is a single bond.

Examples of compounds represented by formula (17) include the nucleoside or nucleotide derivatives represented by formulae (20) to (24). In formulae (20) to (24), R¹² is —H or —OH.

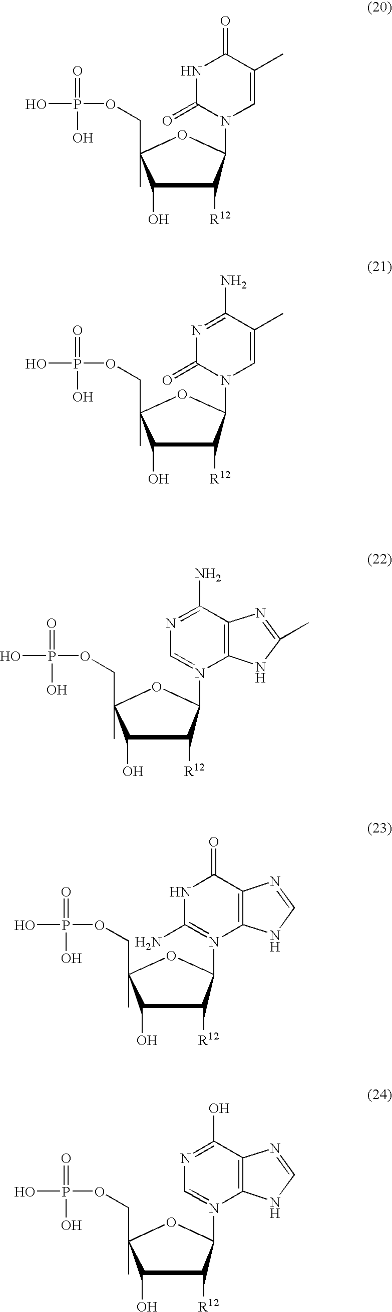

The polynucleotide derivatives of the invention may be DNA or RNA.

The number of nucleotides in the polynucleotide derivatives of the invention is not limited. Such a polynucleotide derivative may have, for example, about 10 to about 100 nucleotides, and preferably about 15 to about 30 nucleotides. The polynucleotides of the invention thus encompass oligonucleotides.

The polynucleotide derivatives of the invention may have about 1 to about 20 nucleotide derivatives of the invention, depending on the total number of nucleotides therein. Preferably, the polynucleotide derivatives have about 1 to about 5 nucleotide derivatives of the invention. When the number of nucleotide derivatives of the invention is within such a range, interaction between their bases does not cause changes in the fluorescence spectrum and shifts in the peak wavelength.

<Intermediate in the Synthesis of Polynucleotide Derivative>

The first intermediate (trityl) of the polynucleotide derivative of the present invention is a novel compound represented by formula (25): 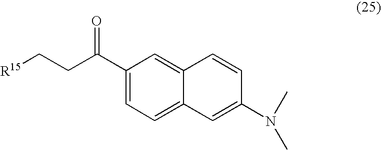
wherein R¹⁵ is a substituent represented by formula (26): 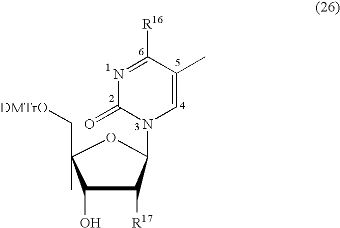
wherein R¹⁷ is —H or —OH; DMTrO— is a dimethoxytrityl group; and R¹⁶ is ═O or —NH₂, with the proviso that when R¹⁶ is ═O, H is attached to the 1-position N of the pyrimidine ring, and the bond between the 1-position N and the 6-position C is a single bond; or a substituent represented by formula (27): 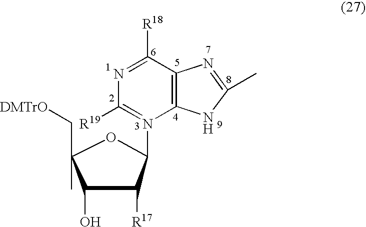
wherein R¹⁷ is —H or —OH; DMTrO— is a dimethoxytrityl group; and R¹⁸ is —OH, ═O, or —NH₂, with the proviso that when R¹⁸ is —OH or —NH₂, R¹⁹ is H; when R¹⁸ is ═O, R¹⁹ is —NH₂; and when R¹⁸ is ═O, H is attached to the 1-position N of the purine ring, and the bond between the 1-position N and the 6-position C is a single bond.

Examples of compounds represented by formula (25) include the compounds represented by formulae (28) to (32). In formulae (28) to (32), R¹⁷ is —H or —OH; and DMTrO— is a dimethoxytrityl group.

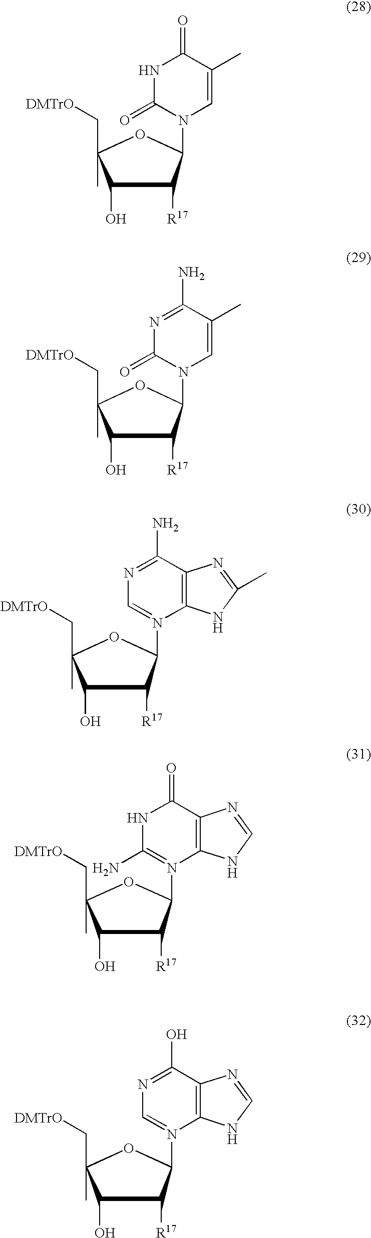

The second intermediate (amidite) of the polynucleotide derivative of the present invention is a novel compound represented by formula (33): 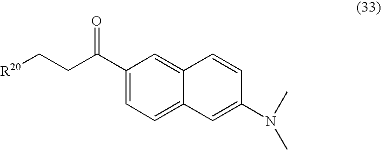
wherein R²⁰ is a substituent represented by formula (34): 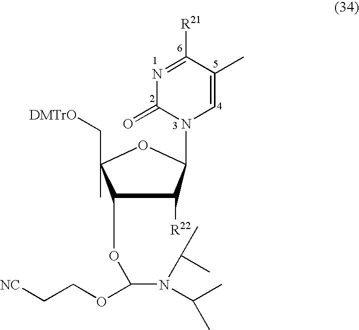
wherein R²² is —H or —OH; DMTrO— is a dimethoxytrityl group; and R²¹ is ═O or —NH₂, with the proviso that when R²¹ is ═O, H is attached to the 1-position N of the pyrimidine ring, and the bond between the 1-position N and the 6-position C is a single bond; or a substituent represented by formula (35): 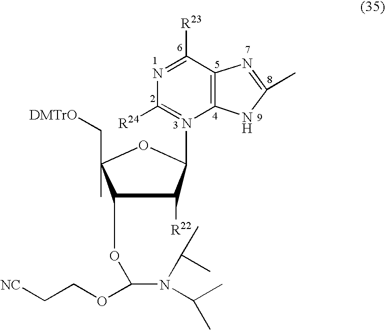
wherein R²² is —H or —OH; DMTrO— is a dimethoxytrityl group; and R³² is —OH, ═O, or —NH₂, with the proviso that when R²³ is —OH or —NH₂, R²⁴ is H; when R²³ is ═O, R²⁴ is —NH₂; and when R²³ is ═O, H is attached to the 1-position N of the purine ring, and the bond between the 1-position N and the 6-position C is a single bond.

Examples of compounds represented by formula (33) include the compounds represented by formulae (36) to (40). In formulae (36) to (40), R²² is —H or —OH; and DMTrO— is a dimethoxytrityl group.

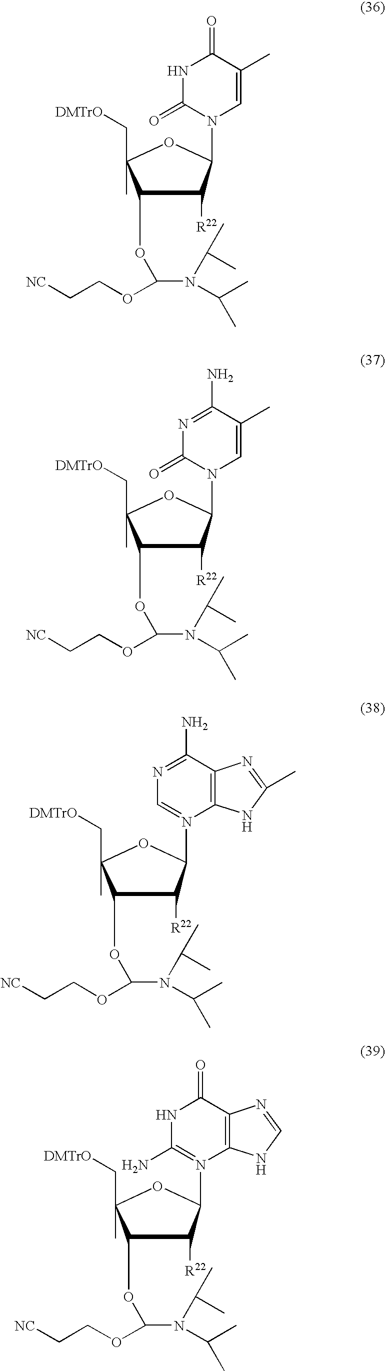

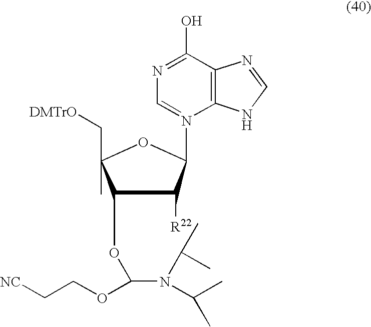

The compound of formula (33) is a nucleoside derivative used as a material in the synthesis of a polynucleotide derivative by the phosphoramidite method. The compound of formula (25) is a synthetic intermediate of the compound of formula (33).

Method of Preparing Compound

<Base Compound>

The base compound (uracil derivative) represented by formula (4) can be obtained by carrying out acid hydrolysis of the N-glycosidic bond of the glycoside of compounds (8) to (12) of the Examples. The base compound (adenine derivative) represented by formula (6) can be obtained by carrying out acid hydrolysis of the N-glycosidic bond of the glycoside of compounds (21) to (24) of the Examples.

The base compound (cytosine derivative) represented by formula (5), the base compound (guanine derivative) represented by formula (7), and the base compound (inosine derivative) represented by formula (8) can be obtained in a similar manner.

<Nucleoside Derivative or Nucleotide Derivative>

The nucleoside derivative (uridine derivative) represented by formula (12) can be synthesized, for example, in the following manner. First, an alcohol is obtained by allowing trimethylsilylacetylene to act on the 6-(N,N-dimethylamino)naphthalene-2-carboxyaldehyde obtainable by a standard method. Subsequently, after removing acetylene-terminal silyl group, introduction into nucleoside is carried out by palladium coupling with silyl-protected 5-iododeoxyuridine. After reducing the triple bond to a single bond by catalytic reduction, the alcohol is oxidized to the ketone. The silyl protecting groups are finally removed, giving a nucleoside derivative represented by formula (12).

The nucleoside derivative (adenosine derivative) represented by formula (14) can be synthesized, for example, in the following manner. First, an alcohol is obtained by allowing trimethylsilylacetylene to act on the 6-(N,N-dimethylamino)naphthalene-2-carboxyaldehyde obtainable by a standard method. Subsequently, after removing acetylene-terminal silyl group, introduction into nucleoside is carried out by palladium coupling with silyl-protected 8-bromodeoxyadenosine. After reducing the triple bond to a single bond by catalytic reduction, the alcohol is oxidized to the ketone. The silyl protecting groups are finally removed, giving a nucleoside derivative represented by formula (14).

The nucleotide derivatives of the invention can be obtained from the corresponding nucleoside derivatives by standard methods. A nucleotide (n=1) can be produced by adding POCl₃ and trimethyl phosphate to a nucleoside derivative. A triphosphate can be produced by adding tributylammonium pyrophosphate to the nucleotide (n=1).

<Intermediate in the Synthesis of Polynucleotide>

The trityls of the nucleoside derivatives represented by formulae (28) and (30), and the amidites of the nucleoside derivatives represented by formulae (36) and (38) can be produced by the methods described in the Examples.

The trityls of the nucleoside derivatives represented by formulae (29), (31) and (32), and the amidites of the nucleoside derivatives represented by formulae (37), (39) and (40) can also be produced in a similar manner.

<Polynucleotide Derivative>

The polynucleotide derivatives of the invention can be chemically synthesized by, for example, the phosphoramidite method with an automated polynucleotide synthesizer, using the amidites of nucleoside derivatives represented by formulae (36) to (40) in place of amidites of nucleosides at particular sites.

Uses

The base compound (compound of formula (1)), nucleoside or nucleotide derivative (compound of formula (9)), and intermediates of polynucleotide derivatives (compounds of formulae (25) and (33)) of the present invention can be used for the synthesis of the polynucleotide derivatives (polynucleotide derivatives containing the compound of formula (17)) of the invention.

The base compound, nucleoside derivative, and nucleotide derivative of the invention fluoresce. In hybridization with a target nucleic acid, the polynucleotide derivative of the present invention, which contains the above compound or derivative, has much greater fluorescence intensity at 490 to 550 nm when the base in the nucleotide that pairs with the polynucleotide derivative is a particular base than when it is any other base.

In hybridization with a target nucleic acid, for example, the polynucleotide derivative containing a uracil derivative of formula (20) has much greater fluorescence intensity at 490 to 550 nm when the base in the nucleotide that pairs with the polynucleotide derivative is adenine than when it is any other base. In hybridization with a target nucleic acid, for example, the polynucleotide derivative containing an adenine derivative of formula (22) has much greater fluorescence intensity at 490 to 550 nm when the base in the nucleotide that pairs with the polynucleotide derivative is thymine than when it is any other base.

Based on these phenomena, the polynucleotide derivative of the invention has the following uses:

(i) A polynucleotide derivative complementary to a target nucleic acid can be used as a probe to determine the kind of nucleotide at a particular position in the target nucleic acid. Examples of target nucleic acids are DNAs and RNAs of humans, animals, plants, viruses, bacteria, fungi, etc. In a similar manner, such a polynucleotide derivative can be used for the detection of single-nucleotide polymorphisms.

(ii) In a DNA chip, the polynucleotide derivative of the invention can be immobilized or adsorbed onto a substrate in place of a polynucleotide to confirm the presence of a specific nucleic acid sequence, and in particular to determine the kind of nucleotide at a particular position in a target nucleic acid.

(iii) When a target nucleic acid containing a particular nucleotide that provides great fluorescence intensity by pairing with the nucleotide derivative of the invention is hybridized with a complementary polynucleotide derivative wherein the nucleotide derivative of the invention is located so as to be paired with the particular nucleotide, then the fluorescence intensity is increased in proportion to the amount of target nucleic acid. Quantitation of a target nucleic acid can be performed based on this phenomenon.

For example, in real-time PCR or real-time Single Strand DNA Amplification (SSDA), the amplification of DNA can be monitored in real time by using, as a probe, a polynucleotide derivative complementary to the target nucleic acid to be amplified. Since DNA amplification rate is proportional to the amount of DNA before amplification, the quantitation of DNA can be thus performed.

Furthermore, in PCR, amplification of DNA can also be monitored in real time by using the polynucleotide derivative of the present invention as a primer. PCR amplification is carried out in the presence of template sequence, thereby synthesizing a double strand nucleic acid amplification product containing a primer formed of the polynucleotide derivative of the invention. In the synthesis of a complementary strand by DNA polymerase, a complementary base is inserted into a position for pairing with the nucleotide derivative of the invention. A completely complementary double strand nucleic acid sequence is thereby formed, and fluorescence intensity is increased.

(iv) Since the polynucleotide derivative of the invention itself is fluorescent, it can be used for the fluorescent labeling of nucleic acids.

(v) The polynucleotide of the invention can be used as a fluorescent probe for Fluorescence In-Situ Hybridization (FISH).

Probe

As mentioned above, the polynucleotide derivative of the present invention can be used as a probe to determine the kind of base at a particular position in a target nucleic acid contained in a sample. This probe, therefore, can be used as a nucleotide identification reagent.

Depending on the kind of sample, the polynucleotide derivative used as a probe may have, for example, about 4 to about 100 nucleotides, and preferably about 10 to about 30 nucleotides.

When the sample is a cell extract or the like that contains nuclease, in order to prevent cleavage by the nuclease, the polynucleotide derivative of the invention may be a modified nucleic acid such as phosphorothioate DNA, H-phosphonate DNA, 2′-O-methyl RNA, phosphorothioate RNA, etc.

In hybridization, the polynucleotide derivative of the invention is usually added to a sample containing a target nucleic acid in an amount of about 1 nM to about 1 mM, and preferably in an amount of about 100 nM to about 50 pM. Depending on the length of polynucleotide, hybridization may be conducted at temperatures of, for example, about 0° C. to about 60° C., particularly about 15° C. to about 40° C., and for a period of, for example, about 5 to about 60 minutes, particularly about 5 to about 10 minutes. The sample for hybridization is adjusted to, for example, a pH of about 5.5 to about 8.0, and particularly about 6.5 to about 8.0.

When hybridizing the polynucleotide derivative of the invention with a target nucleic acid extracted and purified from a sample, hybridization can be carried out under the conditions of, for example, about room temperature (about 25° C.) to about 50° C., for about 10 to about 48 hours, and at about 2-6×SSC, depending on the length of polynucleotide.

Moreover, nucleic acid in a sample can be quantitated by combining the probe containing the polynucleotide derivative of the invention with a nucleic acid amplification method mentioned later. This probe, therefore, can be used as a nucleic acid quantitation reagent.

DNA Chip

In the DNA chip (or DNA array) of the present invention, the polynucleotide derivative of the invention is immobilized or adsorbed onto a substrate. Immobilization includes bonding such as covalent bonding.

The spot size of DNA on the substrate is not limited, and may be, for example, about 50 μm to about 200 μm. The spot pitch thereof is not limited, and may be, for example, about 100 μm to about 500 μm.

The material for the substrate is not limited, and examples thereof include glass, silica, gold, etc. The substrate may be in plate form, bead form, or in any other form.

When bonding the polynucleotide derivative of the invention to a substrate, one end of the polynucleotide derivative can be bonded to the substrate, using a method such as metal-sulfur bonding.

Depending on the kind of sample, the number of polynucleotide derivatives of the invention that are immobilized or adsorbed on a DNA chip may be, for example, about 10 to about 200, and preferably about 50 to about 100.

When the sample is a cell extract or the like that contains nuclease, to prevent cleavage by the nuclease, the polynucleotide derivative of the invention may be a modified nucleic acid such as phosphorothioate DNA, H-phosphonate DNA, 2′-O-methyl RNA, phosphorothioate RNA, etc.

In hybridization, a sample with a concentration of, for example, about 0.1 μM to about 100 μM, and particularly about 1 μM to about 10 μM, can be added to the polynucleotide derivative of the invention immobilized on a substrate. Depending on the kind of polynucleotide derivative, hybridization may be conducted at temperatures of, for example, about 0° C. to about 60° C., particularly about 20° C. to about 40° C., and for a period of, for example, about 1 to about 30 minutes, particularly about 1 to about 5 minutes. The sample for hybridization is adjusted to, for example, a pH of about 5.5 to about 8, and preferably about 6.5 to about 8.

When using a target nucleic acid extracted and purified from a sample, preferably a target nucleic acid dissolved in a hybridization solution (for example, about 2-6×SSC) is allowed to act on the DNA chip of the invention. The time and temperature conditions are similar to those used with non-purified target nucleic acids.

Method of Identifying Nucleotide

The method of identifying a nucleotide according to the present invention comprises the steps of:

hybridizing a polynucleotide derivative of the invention with a target nucleic acid in a sample;

measuring a fluorescence spectrum of the hybridization product; and

comparing the fluorescence spectrum of the hybridization product with a fluorescence spectrum of the polynucleotide derivative before hybridization, thereby identifying a nucleotide at a particular position in the target nucleic acid.

Any sample that contains nucleic acid may be used. Examples thereof include cell extracts, body fluids such as blood, PCR products, oligonucleotides, etc.

The conditions for hybridization are as mentioned above with respect to probes or DNA chips.

The fluorescence spectrum may be measured by standard methods over a wavelength range of about 300 to about 650 nm, and particularly about 450 to about 650 nm. The emission peak is usually observed at about 514 nm. The fluorescence spectrum of the polynucleotide derivative before hybridization and that of a hybridization product are measured, preferably while they are dissolved or suspended in the same medium. The measurement is usually conducted while they are dissolved or suspended in a hybridization solution.

If a probe containing the polynucleotide derivative represented by formula (12) is hybridized with DNA as a target nucleic acid, and the resulting fluorescence intensity near the peak wavelength is, for example, more than twice as great, and preferably more than 3 times as great, as in the case of a single strand, then it can be judged that the base in the nucleotide that pairs with the polynucleotide derivative is adenine.

If such a probe is hybridized with RNA as a target nucleic acid, and the resulting fluorescence intensity near the peak wavelength is, for example, more than twice as great, and preferably more than 3 times as great, as in the case of a single strand, then it can be judged that the base in the nucleotide that pairs with the polynucleotide derivative is adenine.

If a probe containing the polynucleotide derivative represented by formula (14) is hybridized with DNA as a target nucleic acid, and the resulting fluorescence intensity near the peak wavelength is, for example, more than twice as great, and preferably more than 3 times as great, as in the case of a single strand, then it can be judged that the base in the nucleotide that pairs with the polynucleotide derivative is thymine.

When the probe containing the polynucleotide derivative represented by formula (14) is hybridized with RNA as a target nucleic acid, the same results can be obtained as when the target nucleic acid is DNA.

Method of Quantitating Target Nucleic Acid

The method of quantitating a target nucleic acid in a sample according to the present invention comprises the steps of:

hybridizing a polynucleotide derivative of the invention with a target nucleic acid in a sample;

measuring the fluorescence intensity of the hybridization product; and

comparing the fluorescence intensity of the hybridization product with the fluorescence intensity of a hybridization product obtained by hybridizing the above polynucleotide derivative with a target nucleic acid whose concentration is known, thereby determining the concentration of the target nucleic acid.

The fluorescence wavelength to be measured is not limited, and may be near the peak wavelength.

For example, in real-time PCR, amplification of DNA can be monitored in real time by using the polynucleotide derivative of the present invention as a fluorescent probe. Specifically, in the above quantitation method, the polynucleotide of the invention may be used as a probe to be hybridized to the target nucleic acid, which is one strand of amplified double strand DNA. The greater the amount of DNA before amplification is, the smaller is the number of cycles required to obtain high fluorescence intensity. Therefore, DNA can be quantitated. That is, since the speed of increase of fluorescence intensity is proportional to the amount of DNA before amplification, the quantitation of DNA can be performed.

For example, in real-time SSDA, the polynucleotide derivative of the invention may be used as a probe to be hybridized to the target nucleic acid, which is a DNA strand to be amplified. In this case, too, the greater the amount of DNA before amplification is, the smaller is the number of cycles required to obtain high fluorescence intensity. Therefore, DNA can be quantitated.

In quantitation of DNA by PCR or SSDA, the probe using the polynucleotide derivative of the present invention can be designed such that when the nucleotide derivative is a uracil derivative-containing nucleotide, the base that pairs with the nucleotide derivative is adenine. The probe can also be designed such that when the nucleotide derivative is an adenine derivative-containing nucleotide, the base that pairs with the nucleotide derivative is thymine.

Moreover, the amplification of DNA can also be monitored in real time when the polynucleotide derivative of the present invention is used as a primer. DNA can be thus quantitated as when a probe is used.

### EXAMPLES

The present invention is described below in more detail with reference to Examples. However, the scope of the invention is not limited to these examples.

In the following examples, ¹H and ¹³C NMR spectra were measured with Varian Mercury 400 (400 MHz) spectrometer and JEOL JMN α-500 (500 MHz) spectrometer. Coupling constants (J value) are reported in hertz. The chemical shifts are expressed in ppm downfield from tetramethylsilane, using residual chloroform (δ=7.26 in ¹H NMR, δ=77.0 in ¹³C NMR) and methanol (δ=3.30 in ¹H NMR, δ=49.0 in ¹³C NMR) and dimethylsulfoxide (δ=2.48 in ¹H NMR, δ=39.5 in ¹³C NMR) as internal standards.

EI Mass spectra were recorded on JEOL JMS DX-300 spectrometer or JEOL JMS SX-102A spectrometer. FAB Mass spectra were recorded on JEOL JMS DX-300 spectrometer or JEOL JMS HX-110A spectrometer.

Wakogel C-200 was used for silica gel column chromatography. Pre-coated TLC plates Merck silica gel 60 F₂₅₄ was used for monitoring reactions. TLC spots were visualized with UV light or anisaldehyde (a solution of 9.0 ml p-anisaldehyde, 3.5 ml acetic acid and 10 ml sulfuric acid in 330 ml ethanol). All reagents and solvents were used as received. Fluorescence spectra were obtained using a SHIMADZU RF-5300PC spectrofluorophotometer.

Calf intestine alkaline phosphatase (AP) (100 units/ml), snake venom phosphodiesterase (sv PDE) (3 units/ml) and nuclease P1 (P1) were purchased from Boehringer Mannheim. Oligodeoxynucleotides were purchased from Sawady Technology. The reagents for the DNA synthesizer such as A, G, C, T-β-cyanoethyl phosphoramidite, and CPG supports were purchased from Applied Biosystem, or GLEN RESEARCH. Ultrospec 3000pro UV/Visible spectrophotometer (Amarsham Pharmacia Biotec) was used for absorption spectrum measurement.

Masses of oligonucleotides were determined by MALDI-TOF MS (acceleration voltage 21 kV, negative mode) with 2′, 3′, 4′-trihydroxyacetophenone as a matrix, using T8 mer ([M−H]⁻ 2370.61) and T17 mer ([M−H]⁻ 5108.37) as internal standards.

### Example 1

### Synthesis of a PRODAN dU-Containing Polynucleoside Derivative

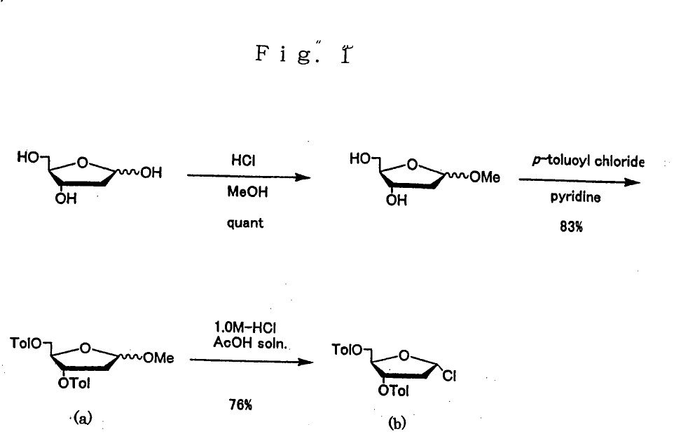

A method of synthesizing a PRODAN dU-containing deoxyribonucleoside derivative, which is one embodiment of the invention, is described with reference to FIG. 1.

<Preparation of a Sugar Portion>

2-deoxy-D-ribose (3.0 g, 22.4 mmol) was dissolved in 0.1% methanol/HCl (60 ml), and the solution was stirred for 20 minutes, giving the methyl glycoside. The solution was neutralized with the addition of silver carbonate (0.88 g, 3.19 ml), and then filtered and concentrated, giving a syrup. The remaining methanol as well as pyridine was removed by distilling twice. The residue was dissolved in anhydrous pyridine, cooled at 0° C., and p-toluoyl chloride (7.50 g, 48.5 mmol) was added dropwise. After the completion of the dropwise addition, the mixture was heated at 50° C. for 2 hours, and stirred at room temperature overnight. Water (100 ml) was added, and the mixture was extracted using ether (200 ml). The ether phase was washed with water, dilute hydrochloric acid (5%), and saturated NaHCO₃ solution, subsequently dried over MgSO₄, and a viscous syrup was obtained by distillation. Pure powdery white 1α/β-O-methyl-2-deoxy-D-ribofuranosyl bis(p-toluate) (FIG. 1, compound (a)) was then obtained by silica gel column chromatography (Wakogel C-200; ethyl acetate:hexane=1:10).

A 1 M hydrochloric acid solution (110 ml) in acetic acid was added to a solution of compound (a) (11.8 g, 30.7 mmol). Gaseous HCl was fed into the solution at room temperature, and after about 10 minutes the chloride crystallized from the solution. The white crystals were collected by filtration, washed with hexane, and dried over potassium hydroxide in a vacuum desiccator, giving 9.0 g of pure 1α-chloro-2-deoxy-D-ribofuranosyl bis(p-toluate) (FIG. 1, compound (b)).

<Introduction of a Nucleic Acid Base Derivative Portion>

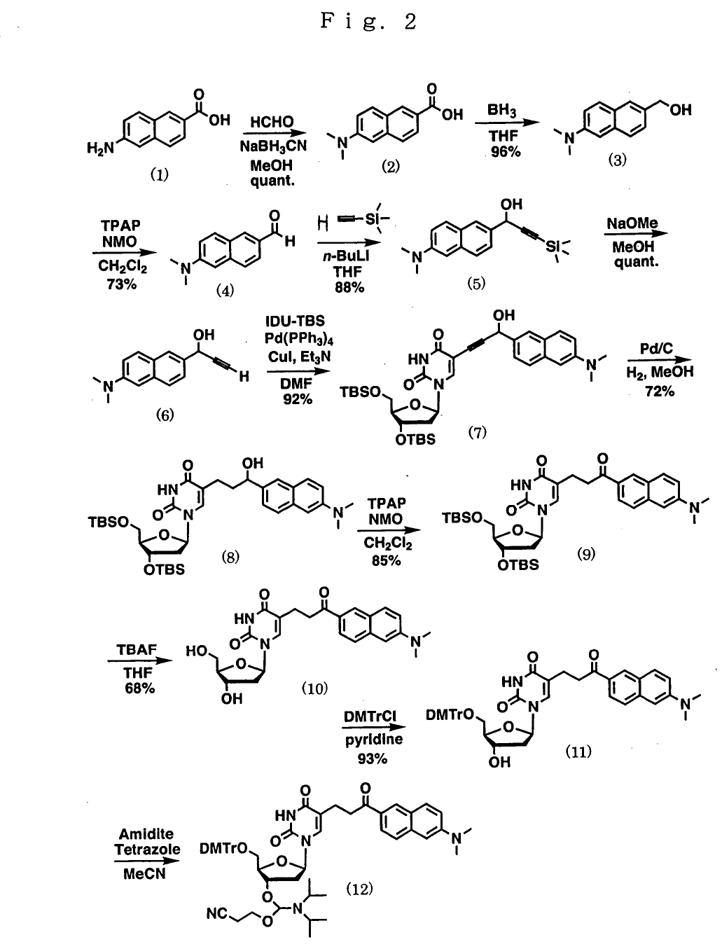

The introduction of a nucleic acid base derivative to the sugar is described with reference to FIG. 2.

### Synthesis of Compound (2) (6-dimethylamino-2-naphtholic acid)

To a solution of 6-amino-2-naphthoic acid (1) (Sigma-Aldrich Japan KK) (90%, 3.00 g, 14.4 mmol) in methanol (120 ml) was added sodium cyanotrihydroborate (3.63 g, 57.7 mmol) and formaldehyde (37% in water, 24 ml), and the reaction mixture was stirred at room temperature for 30 min. After evaporation of the solvent, the residue was extracted with water and chloroform. The aqueous layer was acidified to pH 1 with HCl to give a yellow-ochre solid, and extracted with chloroform. The organic layer was washed with brine, dried over Na₂SO₄, filtered and evaporated in vacuo to yield compound (2) (3.23 g, quant.) as a yellow-ochre solid; ¹H NMR (DMSO-d₆) δ 12.63 (brs, 1H), 8.36 (d, 1H, J=1.5 Hz), 7.88 (d, 1H, J=9.2 Hz), 7.78 (dd, 1H, J=1.8, 8.6 Hz), 7.66 (d, 1H, J=9.0 Hz), 7.26 (dd, 1H, J=2.6, 9.2 Hz), 6.94 (d, 1H, J=2.6 Hz), 3.04 (s, 6H); MS (EI) m/z 215 [M⁺]; HRMS (EI) calculated for C₁₃H₁₃O₂N [M⁺] 215.0946, found 215.0946.

### Synthesis of Compound (3) (6-dimethylamino-2-hydroxymethylnaphthalene)

To a solution of compound (2) (1.37 g, 6.35 mmol) in THF (50 ml) was added borane-THF complex (1M in THF, 25 ml) at 0° C., and the mixture was stirred at room temperature for 1.5 h. After evaporation of the solvent, the reaction was diluted with 0.1 N NaOH aq and extracted with ethyl acetate. The organic layer was washed with brine, dried over Na₂SO₄, filtered and evaporated in vacuo to yield compound (3) (1.22 g, 6.08 mmol, 96%) as a yellow solid; ¹H NMR (CDCl₃) δ 7.70-7.65 (m, 3H), 7.38 (dd, 1H, J=1.9, 8.3 Hz), 7.17 (dd, 1H, J=2.6, 9.0 Hz), 6.94 (s, 1H), 4.77 (s, 2H), 3.05 (s, 6H), 1.79 (brs, 1H); ¹³C NMR (CD₃OD) δ 149.8, 136.4, 135.8, 129.7, 128.5, 127.6, 126.9, 126.4, 117.9, 108.3, 65.6, 41.4; MS (EI) m/z 201 [M⁺]; HRMS (EI) calculated for C₁₃H₁₅ON [M⁺] 201.1154, found 201.1153.

### Synthesis of Compound (4) (6-dimethylamino-2-naphthaldehyde (DAN))

To a solution of compound (3) (654 mg, 3.25 mmol) in dichloromethane (10 ml) was added flame-dried molecular sieves 4 Å (650 mg), and then 4-methylmorpholine N-oxide (571 mg, 4.88 mmol) and tetrapropylammonium perruthenate (228 mg, 0.65 mmol) were added at 0° C. The mixture was stirred at room temperature for 4 h. After the reaction was complete, the mixture was diluted with diethyl ether (20 ml), and magnesium silicate (trade name: florisil; Floridin Co.) (150-250 μm, 60-100 mesh, 1.5 g) was added to the solution and stirred at room temperature for 15 min. The mixture was filtered through celite and washed with diethyl ether, and the filtrate and washings were combined and evaporated under reduced pressure. The residue was extracted with Na₂SO₃ aq and ethyl acetate. The organic layer was washed with brine, dried over Na₂SO₄, filtered and evaporated under reduced pressure. The crude product was purified by silica gel column chromatography (hexane:ethyl acetate=20:1) to yield compound (4) (471 mg, 2.36 mmol, 73%) as a lemon yellow solid; ¹H NMR (CDCl₃) δ 10.00 (s, 1H), 8.15 (s, 1H), 7.84-7.82 (m, 2H), 7.66 (d, 1H, J=8.6 Hz), 7.18 (dd, 1H, J=2.6, 9.2 Hz), 6.89 (d, J=2.6 Hz, 1H), 3.13 (s, 6H); ¹³C NMR (CDCl₃) δ 191.8, 150.6, 138.6, 134.8, 130.7, 130.6, 126.8, 125.0, 123.5, 116.2, 105.5, 40.4; MS (EI) m/z 199 [M⁺]; HRMS (EI) calculated for C₁₃H₁₃ON [M⁺] 199.0997, found 199.0998.

### Synthesis of Compound (5) (6-dimethylamino-2-(3-trimethylsilanyl-1-hydroxypropyl)naphthalene)

To a solution of trimethylsilyl acetylene (0.242 ml, 1.71 mmol) in THF (2 ml) was added 1.6 M n-BuLi (1.03 ml, 1.65 mmol) at −78° C., and stirred at room temperature for 30 min. The mixture was added to compound (4) (273 mg, 1.37 mmol) in THF (3 ml) at −78° C., and stirred at 0° C. for 30 min. After the reaction was quenched with NH₄Cl aq at 0° C. to give a white solid, the mixture was evaporated under reduced pressure. The reaction mixture was extracted with NH₄Cl aq and ethyl acetate. The organic layer was washed with brine, dried over Na₂SO₄, filtered and evaporated under reduced pressure. The crude product was purified by silica gel column chromatography (hexane:ethyl acetate=20-8:1) to yield compound (5) (341 mg, 1.15 mmol, 88%) as a yellow solid; ¹H NMR (CDCl₃) δ 7.80 (s, 1H), 7.69-7.64 (m, 2H), 7.52 (dd, 1H, J=1.7, 8.5 Hz), 7.15 (dd, 1H, J=2.5, 9.1 Hz), 6.91 (s, 1H), 3.03 (s, 6H), 2.33 (brs, 1H), 0.21 (s, 9H); ¹³C NMR (CDCl₃) δ 148.9, 134.8, 133.9, 129.0, 126.8, 126.3, 125.4, 125.1, 116.6, 106.4, 105.3, 91.4, 65.3, 40.9, −0.1; MS (FAB), m/e 297[M⁺]; HRMS (FAB) calculated for C₁₈H₂₃ONSi [M⁺] 297.1549, found 297.1550.

### Synthesis of Compound (6) (6-dimethylamino-2-(1-hydroxypropyl)naphthalene)

A mixture of compound (5) (341 g, 1.15 mmol) and 0.1 M sodium methoxide (11.5 ml) was stirred at room temperature for 1 h. After the reaction was quenched with 1 M HCl, the mixture was evaporated under reduced pressure. The reaction mixture was extracted with water and ethyl acetate. The organic layer was washed with brine, dried over Na₂SO₄, filtered and evaporated in vacuo to yield compound (6) (278 mg, quant.) as a yellow syrup; ¹H NMR (CDCl₃) δ 7.84 (s, 1H), 7.73-7.67 (m, 2H), 7.54 (dd, 1H, J=1.7, 8.5 Hz), 7.18 (dd, 1H, J=2.5, 9.1 Hz), 6.92 (s, 1H), 5.57 (s, 1H), 3.06 (s, 6H), 2.71 (d, 1H, J=2.2 Hz), 2.25 (brs, 1H); ¹³C NMR (CDCl₃) δ 149.0, 134.9, 133.5, 129.0, 126.9, 126.3, 125.3, 124.9, 116.7, 106.3, 83.9, 74.6, 64.7, 40.8; MS (FAB) m/e 225 [M⁺]; HRMS (FAB) calculated for C₁₅H₁₅ON [M⁺] 225.1154, found 225.1150.

### Synthesis of Compound (7) (5-[6-dimethylamino-2-(1-hydroxypropylnyl)naphthalene]-2′-deoxy-3′,5′-O-(tert-butyldimethyl)uridine)

To a solution of IDU-TBDMS (367 mg, 0.630 mmol), tetrakis(triphenylphosphine)palladium (0) (730 mg, 0.63 mmol, 0.3 eq.) and copper (1) iodide (280 mg, 1.4 mmol, 0.67 eq.) in DMF (0.5 ml) were added compound (6) in DMF (278 mg, 1.15 mmol in 3.5 ml), and triethylamine (0.165 ml, 1.15 mmol) and the mixture was stirred at room temperature for 1 h. The mixture was evapolarated in vacuo and diluted with ethyl acetate. The solution was washed with saturated EDTA solution and 5% sodium bisulfite solution, dried over Na₂SO₄, filtered and evaporated under reduced pressure. The crude product was purified by silica gel column chromatography (toluene:ethyl acetate=4:1) to yield a racemic mixture of compound (7) (393 mg, 92%) as a yellow syrup; ¹H NMR (CDCl₃) δ 8.395 (brs, 1H), 8.394 (brs, 1H), 7.98 (d, 2H, J=3.3 Hz), 7.88 (s, 2H), 7.71 (d, 2H, J=9.0 Hz), 7.64 (d, 2H, J=9.6 Hz), 7.56 (dd, 2H, J=1.6, 8.5 Hz), 7.15 (dd, 2H, J=2.5, 9.1 Hz), 6.90 (d, 2H, J=2.0 Hz), 6.27 (dd, 2H, J=5.8, 7.6 Hz), 5.73 (s, 2H), 4.39-4.37 (m, 2H), 3.97-3.96 (m, 2H), 3.85 (ddd, 2H, J=2.6, 5.4, 11.4 Hz), 3.74-3.71 (m, 2H), 3.04 (s, 12H), 2.69 (brs, 2H), 2.30 (ddd, 2H, J=2.5, 5.8, 13.1 Hz), 2.04-1.96 (m, 2H), 0.89-0.83 (m, 36H), 0.08-0.02 (m, 24H); ¹³C NMR (CDCl₃) δ 161.2, 149.0, 142.5, 134.9, 133.7, 129.1, 126.8, 126.42, 126.43, 125.52, 125.46, 125.12, 125.07, 116.6, 106.3, 99.6, 94.2, 88.4, 85.91, 85.88, 77.6, 77.5, 72.4, 65.3, 63.0, 42.0, 40.8, 25.9, 25.7, 18.3, 18.0, −4.8, −4.7, −5.4, −5.6; MS (FAB) m/e 679 [M⁺]; HRMS (FAB) calculated for C₃₆H₅₃O₆N₃Si₂ [M⁺] 679.3473, found 679.3475.

### Synthesis of Compound (8) (5-[6-dimethylamino-2-(1-hydroxypropanoyl)naphthalene]-2′-deoxy-3′,5′-O-(tert-butyldimethyl)uridine)

A solution of compound (7) (673 mg, 0.990 mmol) and 10% Pd/C (186 mg) in methanol was stirred under hydrogen atmosphere for 9 h at room temperature. The mixture was filtered through celite and washed with methanol, and the filtrate and washings were combined and evaporated under reduced pressure. These procedures were repeated on the residue three times. The crude product was purified by silica gel chromatography (hexane:ethyl acetate=3-1:1) to give racemic mixture of compound (8) (488 mg, 0.713 mmol, 72%) as a yellow solid: ¹H NMR (CDCl₃) δ 8.41 (s, 1H), 8.40 (s, 1H), 7.68-7.61 (m, 6H), 7.431 (s, 1H), 7.428 (s, 1H), 7.36 (dd, 1H, J=1.8, 8.4 Hz), 7.35 (dd, 1H, J=1.9, 8.5 Hz), 7.15 (dd, 2H, J=2.5, 9.1 Hz), 6.90 (d, 2H, J=2.4 Hz), 6.30 (dd, 2H, J=5.7, 8.1 Hz), 4.75 (m, 2H), 4.38-4.36 (m, 2H), 3.92 (q, 2H, J=2.5 Hz), 3.79 (ddd, 2H, J=2.9, 4.4, 11.4 Hz), 3.72 (dt, 2H, J=3.0, 11.4 Hz), 3.03 (s, 12H), 2.84 (brs, 1H), 2.75 (brs, 1H), 2.56-2.41 (m, 4H), 2.222 (ddd, 1H, J=2.6, 5.5, 13.0 Hz), 2.216 (ddd, 1H, J=2.7, 5.5, 13.0 Hz), 2.09-1.90 (m, 6H), 0.89-0.85 (m, 36H), 0.08-0.02 (m, 24H); ¹³C NMR (CDCl₃) δ 163.6, 163.5, 149.93, 149.89, 148.7, 137.84, 137.75, 136.21, 136.16, 134.5, 128.7, 126.6, 124.4, 124.3, 124.2, 116.7, 114.6, 106.5, 87.9, 84.99, 84.96, 73.4, 73.3, 72.41, 72.36, 63.1, 63.0, 41.2, 40.9, 38.39, 38.36, 25.90, 25.88, 25.7, 23.9, 23.8, 18.4, 18.0, −4.5, −4.8, −5.40, −5.43, −5.5; MS (FAB) m/e 681 [M⁺]; HRMS (FAB) calculated for C₃₆H₅₅O₆N₃Si₂ [M⁺] 681.3629, found 681.3632.

### Synthesis of Compound (9) (5-PRODAN-2′-deoxy-3′,5′-O-(tert-butyldimethyl)uridine)

To a solution of compound (8) (104 mg, 0.152 mmol) in dichloromethane (4 ml) was added flame-dried molecular sieves (4 Å, 1/16, 100 mg), and then 4-methylmorpholine N-oxide (26.4 mg, 0.225 mmol) and tetrapropylammonium perruthenate (11.0 mg, 0.0313 mmol) were added at 0° C., and the mixture was stirred at 0° C. for 1 h. After the reaction was complete, the mixture was diluted with diethyl ether (8 ml), and magnesium silicate (trade name: florisil; Floridin Co.) (150-250 μm, 60-100 mesh, 100 mg) was added to the solution and stirred at room temperature for 15 min. The mixture was filtered through celite and washed with diethyl ether, and the filtrate and washings were combined and evaporated under reduced pressure. The residue was extracted with Na₂SO₃ aq and ethyl acetate. The organic layer was washed with brine, dried over Na₂SO₄, filtered and evaporated under reduced pressure. The crude product was purified by silica gel column chromatography (hexane:ethyl acetate=4:1) to yield compound (9) (88.1 mg, 0.129 mmol, 85%) as a lemon yellow solid; ¹H NMR(CDCl₃) δ 8.33 (s, 1H), 8.16 (brs, 1H), 7.90 (dd, 1H, J=1.7, 8.7 Hz), 7.78 (d, 1H, J=9.2 Hz), 7.63-7.59 (m, 2H), 7.16 (dd, 1H, J=2.5, 9.1 Hz), 6.86 (d, 1H, J=2.2 Hz), 6.31 (dd, 1H, J=5.9, 7.7 Hz), 4.42 (dt, 1H, J=2.7, 5.7 Hz), 3.94 (q, 1H, J=3.0 Hz), 3.84 (dd, 1H, J=3.6, 11.3 Hz), 3.78 (dd, 1H, J=3.3, 11.2 Hz), 3.41-3.24 (m, 2H), 3.10 (s, 6H), 2.79 (t, 2H, J=7.3 Hz), 2.23 (ddd, 1H, J=2.7, 5.8, 13.1 Hz), 2.02 (ddd, 1H, J=5.9, 7.7, 13.4 Hz), 0.90 (m, 18H), 0.11-0.07 (m, 12H); ¹³C NMR(CDCl₃) δ 198.3, 163.2, 150.2, 150.0, 137.6, 137.2, 130.7, 130.3, 130.0, 126.1, 125.1, 124.4, 116.3, 113.8, 105.3, 87.8, 84.9, 72.3, 63.1, 40.9, 40.4, 36.9, 25.9, 25.7, 22.7, 18.4, 18.0, −4.7, −4.8, −5.3, −5.4; MS (FAB) m/e 682 [(M+H)⁺]; HRMS (FAB) calculated for C₃₆H₅₆O₆N₃Si₂ [(M+H)⁺] 682.3708, found 682.3704.

### Synthesis of Compound (10) (5-PRODAN-2′-deoxyuridine)

To a solution of compound (9) (343 mg, 0.503 mmol) in THF (5 ml) was added a 1 M solution of TBAF (1.2 ml, 1.2 mmol). The mixture was stirred at room temperature for 12 h. The mixture was evaporated under reduced pressure. The crude product was purified by silica gel column chromatography (chloroform:methanol=20:1) to yield compound (10) (15.7 mg, 68%) as a lemon yellow solid; ¹H NMR (DMSO-d₆) δ 11.4 (brs, 1H), 8.46 (s, 1H), 7.89 (d, 1H, J=9.2 Hz), 7.82 (dd, 1H, J=1.7, 8.7 Hz), 7.79 (s, 1H), 7.67 (d, 1H, J=8.8 Hz), 7.27 (dd, 1H, J=2.6, 9.2 Hz), 6.94 (d, 1H, J=2.4 Hz), 6.17 (t, 1H, J=6.9 Hz), 5.27 (brs, 1H), 5.09 (brs, 1H), 4.24 (m, 1H), 3.76 (q, 1H, J=3.5 Hz), 3.61-3.53 (m, 1H), 3.27-3.22 (m, 2H), 3.05 (s, 6H), 2.57 (t, 2H, J=7.6 Hz), 2.14-2.03 (m, 2H); ¹³C NMR (DMSO-d₆) δ 198.2, 163.4, 150.3, 150.1, 137.2, 136.7, 130.6, 129.9, 129.6, 125.9, 124.5, 123.8, 116.4, 112.7, 104.7, 87.3, 83.9, 70.3, 61.2, 39.9, 39.4, 36.6, 21.8; MS (FAB) m/z 454 [(M+H)⁺]; HRMS (FAB) calculated for C₂₄H₂₈O₆ N₃ [(M+H)⁺] 454.1978, found 454.1978.

### Synthesis of Compound (11) (5-PRODAN-2′-deoxy-5′-O-(4,4′-dimethoxytrityl)uridine)

To a solution of compound (10) (140 mg, 0.308 mmol) in pyridine (5 ml) was added 4,4′-dimethoxytrityl chloride (318 mg, 0.937 mmol). The mixture was stirred at room temperature for 4 h. The mixture was evaporated under reduced pressure. The crude product was purified by silica gel column chromatography (hexane:ethyl acetate=1:1.5-2, then ethyl acetate) to yield compound (11) (216 mg, 0.286 mmol, 93%) as a lemon yellow solid; ¹H NMR (DMSO-d₆) δ 11.39 (brs, 1H), 8.31 (s, 1H), 7.83 (d, 1H, J=9.2 Hz), 7.71 (dd, 1H, J=1.6, 8.6 Hz), 7.63 (d, 1H, J=8.8 Hz), 7.53 (s, 1H), 7.36 (d, 2H, J=7.5 Hz), 7.27-7.21 (m, 7H), 7.12 (t, 1H, J=7.3 Hz), 6.93 (d, 1H, J=2.4 Hz), 6.82 (d, 4H, J=8.6 Hz), 6.22 (t, 1H, J=6.8 Hz), 5.30 (brd, 1H, J=4.2 Hz), 4.30 (m, 1H), 3.89-3.88 (m, 1H), 3.64 (s, 6H), 3.20 (d, 2H, J=4.0 Hz), 3.05-3.02 (m, 8H), 2.34-2.16 (m, 4H); ¹³C NMR (DMSO-d₆) δ 197.7, 170.2, 163.2, 158.1, 158.0, 150.2, 150.1, 144.6, 137.1, 136.4, 135.4, 135.3, 130.5, 129.7, 129.5, 127.8, 127.6, 126.6, 125.8, 124.5, 123.7, 116.3, 113.1, 113.0, 104.7, 85.7, 85.5, 83.9, 70.5, 63.8, 54.9, 39.9, 39.3, 36.8, 21.6; MS (FAB) m/e 755 [M⁺]; HRMS (FAB) calculated for C₄₅H₄₅O₈N₃ [M⁺] 755.3207, found 755.3212.

### Synthesis of Compound (12) (5-PRODAN-2′-deoxy-3′-O-(cyanoethyl-N,N′-diisopropylphosphoramidite)-5′-O-(4,4′-dimethoxytrityl)uridine)

To a solution of compound (11) (30.5 mg, 0.0404 mmol) and tetrazole (3.1 mg, 0.0442 mmol) in anhydrous acetonitrile (0.4 ml, for synthesis of nucleic acid) was added 2-cyanoethyl tetraisopropylphosphorodiamidite (13 μl, 0.0398 mmol) under nitrogen. The mixture was stirred at room temperature for 30 min. The mixture was filtered and used with no further purification.

### Example 2

### Synthesis of an oligonucleotide Derivative

Using compound (12) (PRODAN dU amidite) obtained in Example 1 as a material, oligodeoxyribonucleotide derivative 5′-CGCAATnTAACGC-3′ (SEQ ID No. 1) (wherein n is a PRODAN dU-containing deoxyribonucleotide, corresponding to compound (12)) was synthesized by the phosphoramidite method with an Applied Biosystems 392 DNA/RNA synthesizer. An ammonia solution of the obtained oligodeoxyribonucleotide derivative was treated at 55° C. for 5 hours, and vacuum drying was carried out for 20 minutes by a Centrifugal Evaporator (EYELA) to remove ammonia.

The oligodeoxyribonucleotide derivative solution was purified by reverse phase HPLC using a CHEMCOBOND 5-ODS-H column (10×150 mm) (ChemcoPack). Elution was performed with a gradient of 5% to 20% acetonitrile over 30 minutes in 0.1 M triethylamine acetate (TEAA) (pH 7.0) at a flow rate of 3.0 ml/min. In purification, the target compound was detected by measuring absorbance at 254 nm. A freeze dryer (FREEZE DRYER FD-5; product of Tokyo Rikakikai Co., Ltd.) was used to remove solvent from the purified solution, which was then diluted with 0.5 ml of purified water (milliQ water) and freeze-dried again. Triethylamine acetate was thus completely removed and dilution was carried out with 50 μl of milliQ water.

### Example 3

### Identification of an oligonucleotide Derivative

<Mass Spectrometry>

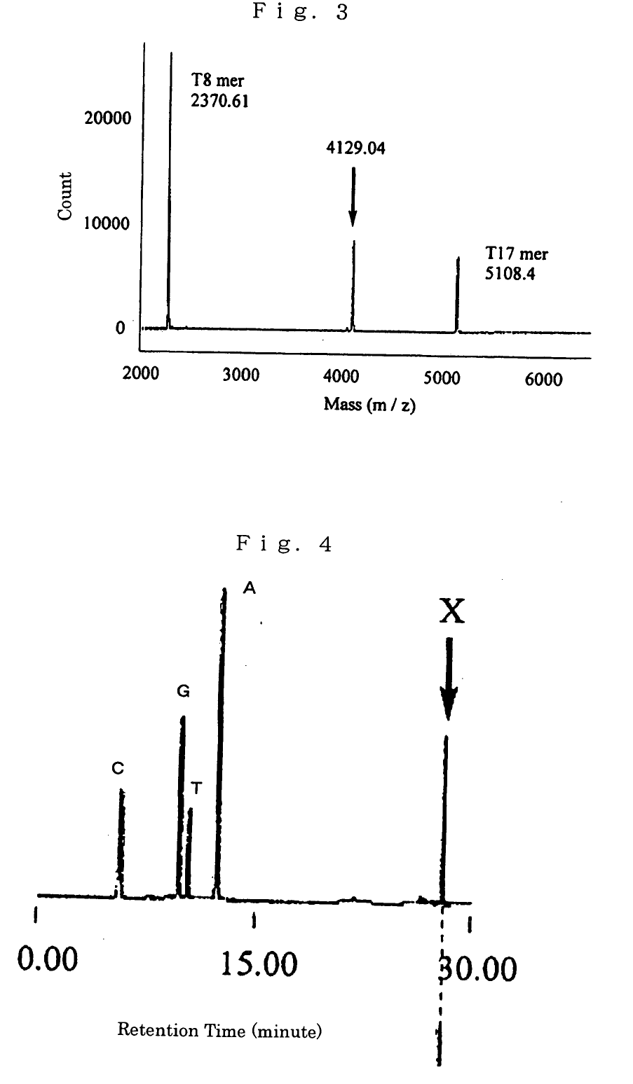

MALDI-TOF mass spectrometry was conducted, using 2′,3′,4′-trihydroxyacetophenone as a matrix, T8mer([M−H]⁻ 2370.61) and T17mer([M−H]⁻ 5108.37) as internal standards, and a spectrophotofluorometer (RF5300PC, product of Shimadzu Corporation). FIG. 3 shows the mass spectrum. [M−H]⁻ calculated as 4128.8321 was detected at 4129.04.

This shows that the oligonucleotide of Example 2 was obtained with the desired composition, without undergoing any side reactions such as degradation, reaction stoppage, etc. in the synthetic process.

<HPLC>

1 μl of sample, 2 μl of mixed enzyme solution (Calf Intestinal Alkaline Phosphatase (100 units/ml; Boehringer Mannheim), snake venom phosphodiesterase I (3 units/ml; Boehringer Mannheim), Nuclease P1 (Boehringer Mannheim)), and 7 μl of milliQ water were mixed and left at 37° C. for 2 hours. The degradation product was analyzed by HPLC using a CHEMCOBOND 5-ODS-H column (4.6×150 mm) (0.1 M trimethylamine acetate (TEAA), pH 7.0, gradient: 0% to 40% acetonitrile over 40 minutes, flow rate: 1.0 ml/min). Using 0.1 mM of deoxyadenylic acid (dA), deoxycytidylic acid (dC), deoxyguanylic acid (dG), and deoxythymidylic acid (dT) as a standard solution, the nucleotides in the PRODAN dU-containing oligodeoxyribonucleotide were confirmed by comparison of peak positions and areas.

The HPLC elution patterns are shown in FIG. 4. In FIG. 4, X shows the elution peak of a sample obtained by the enzymatic degradation of the PRODAN dU-containing oligodeoxyribonucleotide. A, C, G, and T show the elution peaks of the mixture of deoxyadenylic acid (dA), deoxycytidylic acid (dC), deoxyguanylic acid (dG), and deoxythymidylic acid (dT) respectively.

### Example 4

### Fluorescence Analysis

<DNA Derivative>

The oligodeoxyribonucleotide derivative obtained in Example 2 was dissolved to a final concentration of 2.5 μM in a 50 mM phosphate buffer (pH 7.0) containing 0.1 M sodium chloride to prepare a solution. The fluorescence spectrum of the solution was measured at about 25° C. using a spectrophotofluorometer (RF5300PC, product of Shimadzu Corporation). The measurement was carried out at an excitation wavelength of 450 nm and an emission wavelength of 514 nm, with the excitation and emission band widths being 1.5 nm. The fluorescence intensity of the single-strand oligonucleotide derivative was 1.0.

To samples of the above solution was added oligodeoxyribonucleotides (A′): 5′-GCGTTAAATTGCG-3′ (SEQ ID No. 2), (T′): 5′-GCGTTATATTGCG-3+ (SEQ ID No. 3), (G′): 5′-GCGTTAGATTGCG-3′ (SEQ ID No. 4), or (C′): 5′-GCGTTACATTGCG-3′ (SEQ ID No. 5), which are complementary to the oligodeoxyribonucleotide derivative (SEQ ID No. 1) in the portions other than the nucleotide derivative (n), to a final concentration of 2.5 μM. Each solution sample was then mixed with a vortex mixer.

The fluorescence spectra of the solution samples were measured using the spectrophotofluorometer. When oligodeoxyribonucleotide (A′) was added, the fluorescence intensity at 514 nm was 5.0. When oligodeoxyribonucleotide (T′) was added, the fluorescence intensity at 514 nm was 2.1. When oligodeoxyribonucleotide (G′) was added, the fluorescence intensity at 514 nm was 2.0. When oligodeoxyribonucleotide (C′) was added, the fluorescence intensity at 514 nm was 2.0.

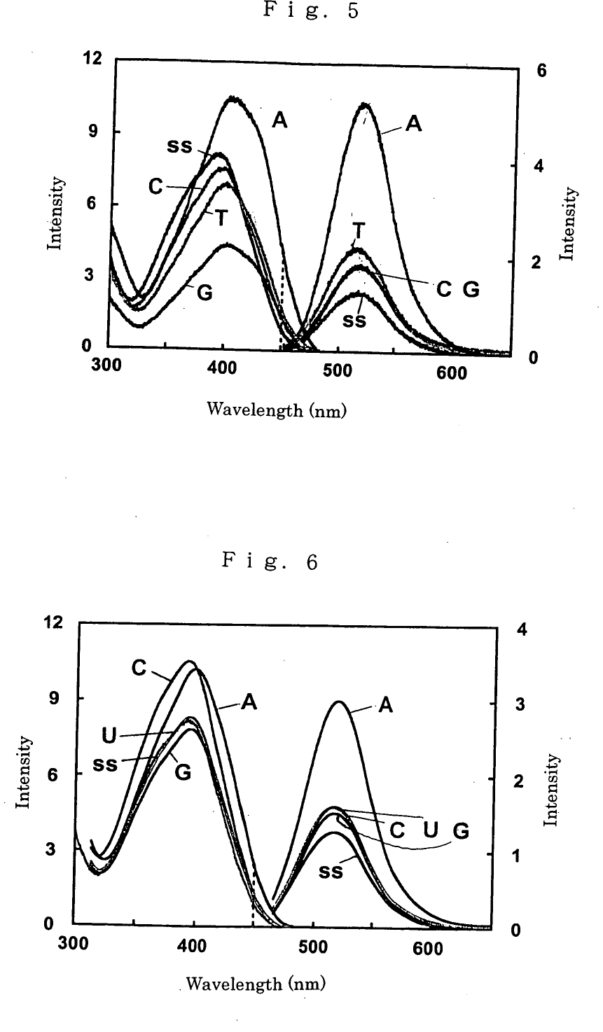

As is clear from the above, when the nucleotide on the complementary strand that paired with the nucleotide derivative (n) of the oligodeoxyribonucleotide derivative (SEQ ID No. 1) was deoxyadenylic acid, the fluorescence intensity was increased to 5 times the original intensity by hybridization. This degree of increase is much higher than in the cases of the other nucleotides. FIG. 5 shows the fluorescence spectra. In FIG. 5, the vertical axis scale on the left shows absorbance for the absorption spectra, and the vertical axis scale on the right shows the fluorescence intensity of the fluorescence spectra.

<RNA Derivative>

The oligodeoxyribonucleotide derivative obtained in Example 2 was dissolved to a final concentration of 2.5 μM in a 50 mM phosphate buffer (pH 7.0) containing 0.1 M sodium chloride to prepare a solution. The fluorescence spectrum of the solution was measured at about 25° C. using a spectrophotofluorometer (RF5300PC, product of Shimadzu Corporation). The measurement was carried out at an excitation wavelength of 450 nm and an emission wavelength of 514 nm, with the excitation and emission band widths being 1.5 nm. The fluorescence intensity of the single-strand oligonucleotide derivative was 1.2.

To samples of the above solution was added oligoribonucleotides (A′): 5′-GCGUUAAAUUGCG-3′ (SEQ ID No. 6), (U′): 5′-GCGUUAUAUUGCG-3× (SEQ ID No. 7), (G′): 5′-GCGUUAGAUUGCG-3′ (SEQ ID No. 8), or (C′): 5′-GCGUUACAUUGCG-3′ (SEQ ID No. 9), which are complementary to the oligodeoxyribonucleotide derivative (SEQ ID No. 1) in the portions other than the nucleotide derivative (n), to a final concentration of 2.5 μM. Each solution sample was then mixed with a vortex mixer.

The fluorescence spectra of the solution samples were measured using the spectrophotofluorometer. When oligoribonucleotide (A′) was added, the fluorescence intensity at 514 nm was 3.0. When oligoribonucleotide (U′) was added, the fluorescence intensity at 514 nm was 1.6. When oligoribonucleotide (G′) was added, the fluorescence intensity at 514 nm was 1.5. When oligoribonucleotide (C′) was added, the fluorescence intensity at 514 nm was 1.6.

As is clear from the above, when the nucleotide on the complementary strand that paired with the nucleotide derivative (n) of the oligodeoxyribonucleotide derivative (SEQ ID No. 1) was adenylic acid, the fluorescence intensity was increased to about 3 times the original intensity by hybridization. This degree of increase is much higher than in the cases of the other nucleotides. FIG. 6 shows the fluorescence spectra. In FIG. 6, the vertical axis scale on the left shows absorbance for the absorption spectra, and the vertical axis scale on the right shows the fluorescence intensity of the fluorescence spectra.

### Example 5

### Detection of Gene Polymorphism and Quantitation of DNA by Real-Time SSDA using the Probe of the Present Invention

Gene polymorphism at location *3 of CYP2C19 as a drug-metabolizing enzyme P-450 was detected by a single strand DNA amplifying method (SSDA) using the polynucleotide derivative of the present invention.

5′-AGATTAATGTAAAAGTGATGTGTTGATTTTATGCATGCCA-3′ (SEQ ID No. 10) was used as a forward primer; 5′-GGGCTTGGTCAATATAGAATTTTGGATTTCCC-3′ (SEQ ID No. 11) was used as a reverse primer; and 5′-GTGATCTGCTCCATTATTTT-3′ (SEQ ID No. 12) was used as an inner primer having a low melting temperature. These constitute a primer set to specifically amplify the CYP2C19*3 polymorphic gene including the region of the variation of the 636-position G being A.

Compound (12) (PRODAN dU amidite) obtained in Example 1 was used as a material, following the method of Example 2, to obtain 5′-CTGGATnCAGGGGGTGCTTAC-3′P (SEQ ID No. 13) probe (PRODAN dU probe) wherein n is a PRODAN dU deoxyribonucleotide derivative and P is phosphate group. This probe is complementary to the partial sequence of the DNA strand elongated by the inner probe, and has a PRODAN dU nucleotide derivative in the position corresponding to the 636th base of CYP2C19*3.

Three kinds of zygotes were used as template DNAs: a homozygote wherein the 636th base of CYP2C19*3 was G, a homozygote having the variation of the 636th base to A, and a heterozygote (G/A). DNA was extracted from cell strain K562, using a QIAamp DNA Mini Kit, and the target sequence was amplified under the following conditions. The amplification fragment was inserted into a pCR2.1 vector and was used as the G homozygote control plasmid. The A homozygote control plasmid was obtained by variation introduction into a G homozygote control plasmid using a site-directed mutagenesis method. The heterozygote control plasmid was produced by mixing G homozygote control plasmid and A homozygote control plasmid in equal quantities.

A reaction solution (20 μl) containing forward primer (800 nM), reverse primer (400 nM), inner primer (800 nM), PRODAN dU probe (250 nM), tricine-KOH (pH 8.0) (40 mM), KCl (16 mM), MgCl₂ (4.5 mM), BSA (3.75 μg/ml), dNTPs (each 600 nM), 1× TITANIUM Taq PCR buffer, and template DNA (1 μl) was prepared. This was subjected to real-time SSDA using a real-time PCR instrument Light Cycler (Ex/Em=470/530) (Roche) under the conditions of 95° C. for 1 minute; 20 cycles of 95° C. for 10 seconds and 68° C. for 30 seconds; 50 to 60 cycles of 95° C. for 10 seconds, 55° C. for 10 seconds and 68° C. for 30 seconds; and 68° C. for 3 minutes.

Six kinds of samples prepared by graded dilution to 10¹, 10², 10³, 10⁴, 10⁵, and 10⁶ copies/μl were used for the template DNA.

During annealing at 55° C., fluorescence intensity was detected under the conditions of single mode, excitation wavelength: 470 nm (LED), detection filter: channel 1 (530 nm), and fluorimeter gains 75.

The results are shown in FIG. 7. In FIG. 7, NTC indicates a control that does not use template DNA (No Template Control). The fluorescence intensity obtained by the homozygote whose 636th base was A was about 7 times as great as that obtained by the homozygote whose 636th base was G. The higher the template DNA concentration was, the smaller was the number of cycles required to increase fluorescence intensity. Therefore, it is clear that DNA can be quantitated by this method.

### Example 6

### Detection of Gene Polymorphism and Quantitation of DNA by Real-Time PCR using the Probe of the Present Invention

Instead of using the forward primer (SEQ ID No. 10) of Example 5, the inner primer (SEQ ID No. 12) was used as a forward primer.

The same operation as in Example 5 was performed except that the concentrations in the reaction solution were forward primer (SEQ ID No. 12): 1000 nM, reverse primer (SEQ ID No. 11): 200 nM, probe (SEQ ID No. 13): 500 nM, MgCl₂: 3.5 mM, dNTPs: each 200 nM; and that the reaction temperature conditions were 95° C. for 1 minute and 40 cycles of 95° C. for 10 seconds, 55° C. for 10 seconds and 68° C. for 30 seconds.

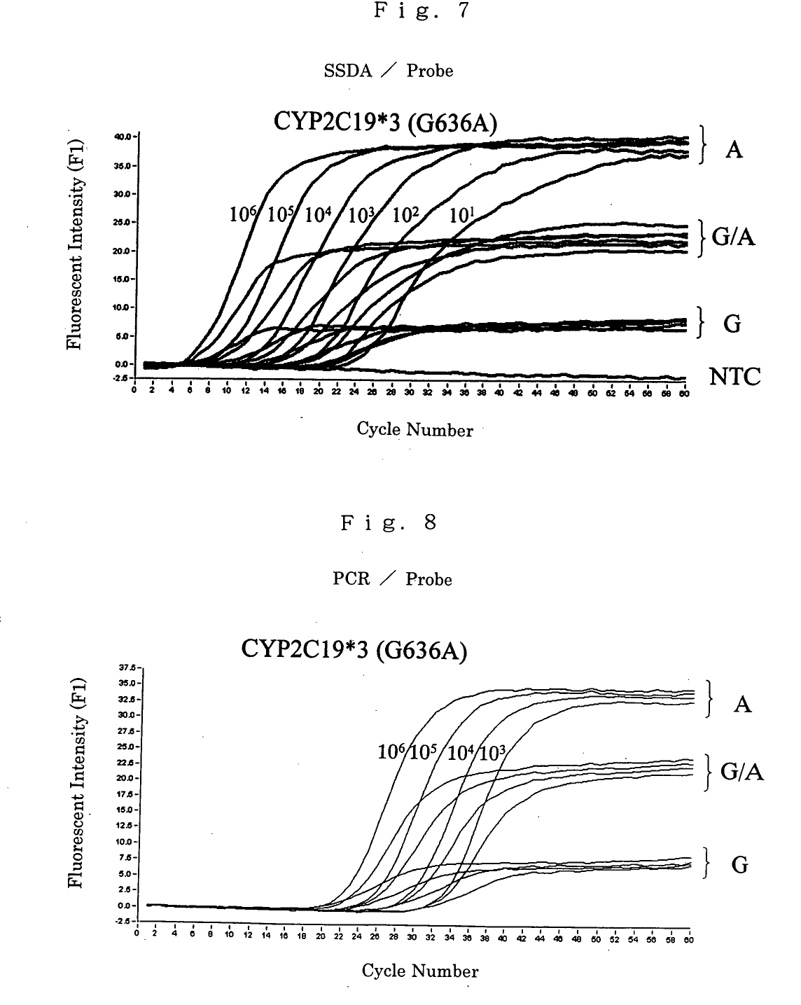

The results are shown in FIG. 8. The fluorescence intensity obtained by the homozygote whose 636th base was A was about 5 times as great as that obtained by the homozygote whose 636th base was G. The higher the template DNA concentration was, the smaller was the number of cycles required to increase fluorescence intensity. Therefore, it is clear that DNA can be quantitated by this method.

### Example 7

### Quantitation of Bacterial DNA by Real-Time PCR using the Primer of the Present Invention

DNA was extracted from Mycoplasma pneumoniae ATCC 29342 strain and Streptococcus pneumoniae ATCC 6303 strain, using a QIAamp UltraSense Virus Kit. The target sequences in the extracted DNAs were amplified by the method below, and inserted into pCR2.1 to be used as control plasmids.

With respect to the Mycoplasma pneumoniae DNA, 5′-CTCCATCAAGCTnTCGCT-3′ (SEQ ID No. 14; wherein n is a PRODAN dU-containing deoxyribonucleotide) was used as a forward primer; and 5′-CGAAAGTAGTAATACTTTAGAGGCG-3′ (SEQ ID No. 15) was used as a reverse primer. With respect to the Streptococcus pneumoniae DNA, 5′-GAAGAAGACTATGCTCGnAGATCAGA-3′ (SEQ ID No. 16; wherein n is a PRODAN dU-containing deoxyribonucleotide) was used as a forward primer; and 5′-CATCAGTATTGTAGAAGTACCACATACC-3′ (SEQ ID No. 17) was used as a reverse primer. The forward primer has a PRODAN dU nucleotide derivative in the position corresponding to a specific A in the template DNA. The forward primer was synthesized, using compound (12) (PRODAN dU amidite) obtained in Example 1 as a material and following the method of Example 2.

For Mycoplasma pneumoniae detection, a reaction solution (20 μl) containing forward primer (600 nM), reverse primer (600 nM), tricine-KOH (pH 8.0) (40 mM), KCl (16 mM), MgCl₂ (3.5 mM), BSA (3.75 μg/ml), dNTPs (each 200 nM), 1× TITANIUM Taq PCR buffer, and template DNA (1 μl) was prepared. This was subjected to real-time PCR using a Light Cycler (Ex/Em=470/530) under the conditions of 95° C. for 1 minute; and 40 cycles of 95° C. for 10 seconds, 55° C. for 10 seconds and 68° C. for 30 seconds.

For Streptococcus pneumoniae detection, the same operation as in Mycoplasma pneumoniae detection was performed except that the concentrations of forward primer and reverse primer were each 400 nM and that the control plasmid containing Streptococcus pneumoniae DNA was used for the template DNA.

Five kinds of samples prepared by graded dilution to 10¹, 10², 10³, 10⁴, and 10⁵ copies/μl were used for the template DNA.

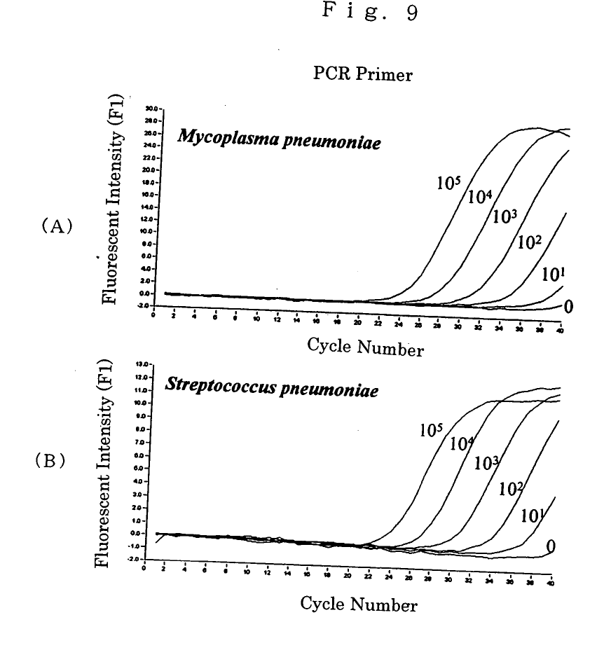

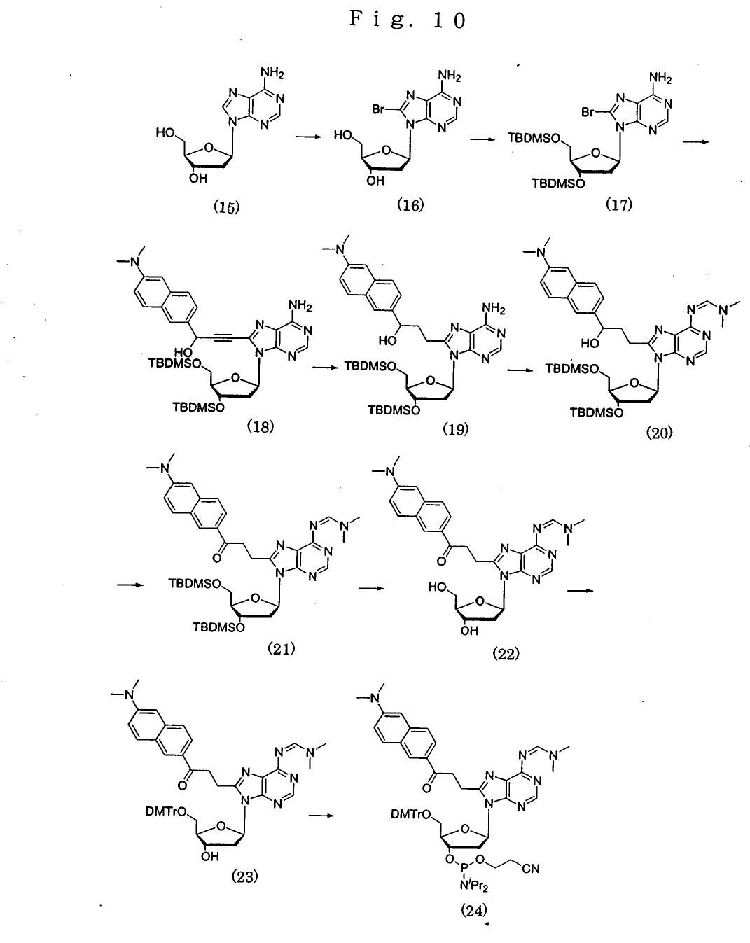

The results are shown in FIG. 9. FIG. 9(A) shows the results for Mycoplasma pneumoniae, and FIG. 9(B) shows the results for Streptococcus pneumoniae. The higher the DNA concentration was, the smaller was the number of cycles required to increase fluorescence intensity. Therefore, it is clear that DNA can be quantitated by this method.

### Example 8

### Synthesis of a PRODAN dA-Containing Polynucleoside Derivative

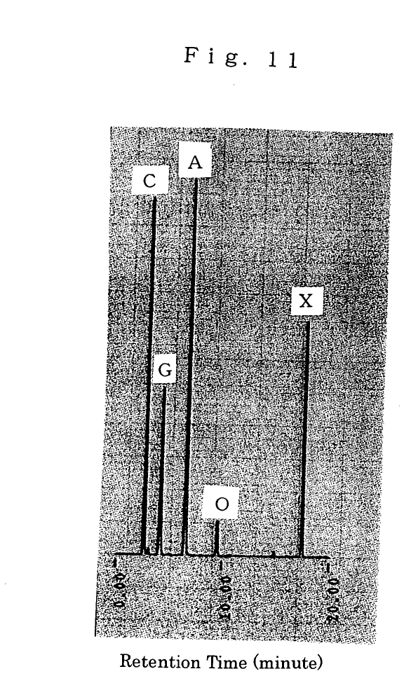

A method of synthesizing a PRODAN dA-containing polydeoxyribonucleotide derivative, which is one embodiment of the invention, is described with reference to FIG. 10.

### Synthesis of Compound (16) (8-bromo-2′-deoxyadenosine)

To a solution of deoxyadenosine (15) (3 g, 11 mmol) in methanol (50 ml) was added NBS (2.6 g, 14.6 mmol, 1.3 eq.). The mixture was stirred at 40° C. for 18 h. The pink suspension was filtered, and the filtrate was dried in vacuo to yield compound (16) (900 mg, 25%) as a pink solid.

### Synthesis of Compound (17) (8-bromo-2′-deoxy-3′,5′-O-(tert-butyldimethylsilyl)adenine)

To a solution of 8-bromo-2′-deoxyadenosine (900 mg, 2.73 mmol) in DMF (15 ml) was added tert-butyldimethylchlorosilane (1.23 g, 8.16 mmol, 3 eq.) and imidazole (0.56 g, 8.23 mmol, 3 eq.), and the mixture was stirred at room temperature for 3 h. After evaporation of the solvent, the residue was extracted with water and chloroform. The organic layer was washed with brine, dried over MgSO₄, filtered and evaporated in vacuo. The crude product was purified by silica gel column chromatography (chloroform) to yield to compound (17) (2.9 g, quant.) as a pink solid.

### Synthesis of Compound (18) (8-[6-dimethylamino-2-(1-hydroxypropynyl)naphthalene]-2′-deoxy-3′,5′-O-(tert-butyldimethyl)adenine)

To a solution of compound (17) (1.9 g, 3.4 mmol, 1.1 eq), tetrakis(triphenylphosphine)palladium (0) (718 mg, 0.62 mmol, 0.2 eq.), copper (1) iodide (240 mg, 1.26 mmol, 0.4 eq.) and compound (6) (0.7 g, 3.1 mmol.) of FIG. 2 in DMF (30 ml) was added triethylamine (1.43 ml, 10 mmol, 3.3 eq.), and the mixture was stirred at room temperature for 17 h. The mixture was evapolarated in vacuo and diluted with ethyl acetate. The solution was washed with saturated EDTA solution and 5% sodium bisulfite solution, dried over Na₂SO₄, filtered and evaporated under reduced pressure. The crude product was purified by silica gel column chromatography (chloroform:methanol=50:1) to yield a racemic mixture of compound (18) (1.33 g, 61%) as a brown solid; ¹H NMR (CDCl₃) δ=8.27 (s, 1H), 7.90 (brs, 1H), 7.70 (d, 1H, J=8.8 Hz), 7.66 (dd, 1H, J=2.0, 8.8 Hz), 7.56-7.60 (m, 1H), 7.16 (dd, 1H, J=2.4, 9.2 Hz), 6.89 (d, 1H, J=2.8 Hz), 6.49 (t, 1H, J=6.4 Hz), 5.85 (d, 1H, J=6.8 Hz), 5.82 (s (br), 2H), 4.78 (ddd, 1H, J=3.6, 5.6, 17.6 Hz), 3.86-3.97 (m, 2H), 3.67 (ddd, 1H, J=2.4, 4.8, 10.4 Hz), 3.46 (quin., 1H, J=6.0 Hz), 3.05 (s, 6H), 2.20 (ddd, 1H, J=4.0, 6.8, 17.2 Hz), 0.89 (s, 9H), 0.83 (s, 9H), 0.10 (s, 3H), 0.087 (s, 3H), 0.006 (s, 3H), 0.037 (s, 3H); ¹³C NMR (CDCl₃) δ=155.0, 153.3, 149.4, 149.1, 135.1, 134.8, 132.5, 129.1, 127.12, 127.07, 126.3, 125.8, 125.6, 124.89, 124.85, 116.7, 106.2, 87.87, 87.84, 85.2, 72.6, 65.1, 65.0, 63.0, 40.8, 37.3, 18.4, 18.0, −4.7, −4.8, −5.4, −5.5; MS (FAB, NBA/CHCl₃) m/z 702 [(M+H)⁺]; HRMS (FAB) calculated for C₃₇H₅₄ N₆O₄Si₂ [(M+H)⁺] 702.3745, found 702.3763.

### Synthesis of Compound (19) (8-[6-dimethylamino-2-(1-hydroxypropanoyl)naphthalene]-2′-deoxy-3′,5′-O-(tert-butyldimethyl)adenine)

A solution of compound (18) (1.3 g, 1.9 mmol) and 5% Pd/C (1.5 g) in methanol was stirred under hydrogen atmosphere for 2 days at room temperature. The mixture was filtered through celite and washed with methanol, and the filtrate and washings were combined and evaporated under reduced pressure. The crude product was purified by silica gel chromatography (chloroform:methanol=50:1) to give racemic mixture of compound (19) (604 mg, 45%) as a light yellow solid: ¹H NMR (CDCl1₃) δ=8.24 (s, 1H), 7.67 (d, 2H, J=8.8 Hz), 7.64 (d, 1H, J=8.4 Hz), 7.37 (dd, 1H, J=1.6, 8.4 Hz), 7.16 (dd, 1H, J=2.4, 9.2 Hz), 6.91 (d, 1H, J=2.4 Hz), 6.20 (dt, 1H, J=6.8 Hz), 5.57 (s (br), 2H), 4.95 (dt, 1H, J=6.0, 22.0 Hz), 4.77 (dt, 1H, J=3.6, 6.0 Hz), 3.83-3.93 (m, 2H), 3.54-3.67 (m, 2H), 3.07-3.13 (m, 2H), 3.04 (s, 6H), 2.36-2.44 (m, 2H), 2.11-2.19 (m, 1H), 0.621 (d, 9H, J=2.8 Hz), 0.812 (d, 9H, J=4.8 Hz), 0.121 (s, 6H), −0.062-−0.016 (m, 6H); MS (FAB, NBA/CHCl₃) m/z 707 [(M+H)⁺]; HRMS (FAB) calculated for C₃₇H₅₈O₄N₆Si₂ [(M+H)⁺] 706.4058, found 707.4135.

### Synthesis of Compound (20) ((dimethylaminomethylidene)amino-8-[6-dimethylamino-2-(1-hydroxypropanoyl)naphthalene]-2′-deoxy-3′,5′-O-(tert-butyldimethyl)adenine)

To a solution of compound (19) (604 mg, 0.85 mmol) in DMF (5 ml) was added DMF dimethyl acetal (6.8 ml, 50 mmol, 59 eq.), and the mixture was stirred at room temperature for 6 h. After evaporation of the solvent, the residue was purified by silica gel chromatography (chloroform) to give a racemic mixture of compound (20) (483 mg, 75%) as a brown oil: ¹H NMR (CDCl₃) δ=8.89 (s, 1H), 8.45 (s, 1H), 7.67 (s, 1H), 7.64 (d, 1H, J=4.4 Hz), 7.62 (s, 1H), 7.38 (dt, 1H, J=1.2, 8.8 Hz), 7.15 (dd, 1H, J=2.4, 9.2 Hz), 6.91 (d, 1H, J=2.4 Hz), 6.25 (dt, 1H, J=6.8, 22.4 Hz), 4.93-5.00 (m, 1H), 4.78 (s (br), 1H), 3.86-3.92 (m, 2H), 3.53-3.68 (m, 2H), 3.24 (s, 3H), 3.18 (d, 3H, J=1.2 Hz), 3.05-3.16 (m, 2H), 3.03 (s, 6H), 2.37-2.48 (m, 2H), 2.12-2.19 (m, 1H), 1.25-1.44 (m, 1H), 0.92 (d, 9H, J=2.0 Hz), 0.822 (d, 9H, J=2.8 Hz), 0.12 (d, 6H, J=2.0 Hz), 0.057-0.016 (dd, 6H, J=3.6, 14.4 Hz); MS (FAB, NBA/CHCl₃) m/z 762 [(M+H)⁺]; HRMS (FAB) calculated for C₄₀H₆₃O₄N₇Si₂ [(M+H)⁺] 761.4480, found 762.4568.

### Synthesis of Compound (21) ((dimethylaminomethylidene)amino-8-DAN-2′-deoxy-3′,5′-O-(tert-butyldimethyl)adenine)

To a solution of compound (20) (116 mg, 0.15 mmol) in dry dichloromethane (5 ml) was added flame-dried molecular sieves (4 Å, 175 mg), and then 4-methylmorpholine N-oxide (26 mg, 0.225 mmol, 1.5 eq.) and tetrapropylammonium perruthenate (5 mg, 0.014 mmol, 0.1 eq.) were added at 0° C., and the mixture was stirred at 0° C. for 15 h. After the reaction was complete, the mixture was diluted with diethyl ether (50 ml) and magnesium silicate (trade name: florisil) (150-250 μm, 60-100 mesh, 175 mg) was added to the solution and stirred at room temperature for 15 min. The mixture was filtered through celite and washed with diethyl ether, and the filtrate and washings were combined and evaporated under reduced pressure. The crude product was purified by silica gel column chromatography (chloroform) to yield compound (21) (37 mg, 32%) as a yellow solid; ¹H NMR (CDCl₃) δ=8.85 (s, 1H), 8.45 (s, 1H), 8.44 (d, 1H, J=1.2 Hz), 7.98 (dd, 1H, J=2.0, 8.8 Hz), 7.80 (d, 1H, J=8.8 Hz), 7.64 (d, 1H, J=8.8 Hz), 7.17 (dd, 1H, J=2.4, 9.2 Hz), 6.87 (d, 1H, J=2.4 Hz), 6.44 (t, 1H, J=7.2 Hz), 4.83 (quin., 1H, J=2.4 Hz), 3.62-3.97 (m, 5H), 3.41-3.47 (m, 2H), 3.20 (s, 3H), 3.11 (s, 6H), 2.99 (s, 3H), 2.27 (ddd, 1H, J=4.0, 6.8, 13.2 Hz), 0.94 (s, 9H), 0.82 (s, 9H), 0.15 (d, 6H, J=1.2 Hz), 0.029 (d, 6H, J=15.2 Hz); ¹³C NMR (CDCl₃) δ=197.8, 158.1, 154.5, 152.96, 151.4, 150.3, 137.7, 130.7, 130.3, 130.1, 126.2, 125.3, 125.1, 124.5, 116.3, 105.3, 87.5, 84.1, 72.5, 62.9, 41.0, 40.4, 37.4, 35.5, 35.1, 25.9, 22.7, 18.3, 18.1, −4.6, −4.7, −5.4, −5.5; MS (FAB, NBA/CHCl₃) m/z 760 [(M+H)⁺]; HRMS (FAB) calculated for C₄₀H₆₁O₄N₇Si₂ [(M+H)⁺] 759.4324, found 760.4403.

### Synthesis of Compound (22) ((dimethylaminomethylidene)amino-8-DAN-2′-deoxyadenine)

To a solution of compound (21) (37 mg, 0.049 mmol) in THF (500 μl) was added a 1 M solution of TBAF (200 μl, 0.2 mmol, THF (500 μl) was added a 1 M solution of TBAF (200 μl, 0.2 mmol, 2.5 eq.) . The mixture was stirred at room temperature for 3 h. The mixture was evaporated under reduced pressure. The crude product was purified by silica gel column chromatography (chloroform:methanol=50:1) to yield compound (22) (24 mg, 92%) as a lemon yellow solid; ¹H NMR (CDCl₃) δ=8.87 (s, 1H), 8.44 (s, 1H), 8.42 (d, 1H, J=1.6 Hz), 7.95 (dd, 1H, J=1.6, 8.8 Hz), 7.79 (d, 1H, J=9.2 Hz), 7.63 (d, 1H, J=8.8 Hz), 7.16 (dd, 1H, J=2.4, 7.6 Hz), 6.86 (d, 1H, J=2.8 Hz), 6.57 (dd, 1H, J=5.6, 9.6 Hz), 4.84 (d, 1H, J=5.2 Hz), 4.25 (s, 1H), 3.70-4.03 (m, 6H), 3.25-3.41 (m, 3H), 3.23 (s, 3H), 3.11 (s, 6H), 3.04 (s, 3H), 2.37 (dd, 1H, J=6.0, 12.0 Hz); ¹³C NMR (CDCl₃) δ=197.5, 159.0, 158.3, 153.2, 151.8, 151.0, 150.3, 137.8, 130.8, 130.2, 130.0, 126.2, 125.0, 124.4, 116.3, 105.2, 89.5, 86.3, 73.8, 63.6, 41.2, 40.4, 35.5, 35.2, 24.1, 22.4, 20.2, 19.7; MS (FAB, DTT/TG/CHCl₃) m/z 532 [(M+H)⁺]; HRMS (FAB) calculated for C₂₈H₃₃O₄N₇ [(M+H)⁺] 531.2594, found 532.2683.

### Synthesis of Compound (23) ((dimethylaminomethylidene)amino-8-DAN-2′-deoxy-5′-O-(4,4′-dimethoxytrityl)adenine)

To a solution of compound (22) (49 mg, 0.092 mmol) in pyridine (3 ml) was added 4,4′-dimethoxytrityl chloride (37 mg, 0.11 mmol, 1.2 eq.). The mixture was stirred at room temperature for 3 h. The mixture was evaporated under reduced pressure. The crude product was purified by silica gel column chromatography (chloroform:methanol=50:1) to yield compound (23) (37 mg, 48%) as a lemon yellow solid; ¹H NMR (CDCl₃) δ=8.82 (s, 1H), 8.39 (d, 1H, J=1.2 Hz), 8.30 (s, 1H), 7.93 (dd, 1H, J=2.0, 8.8 Hz), 7.74 (d, 1H, J=9.2 Hz), 7.60 (d, 1H, J=8.4 Hz), 7.38 (d, 2H, J=7.2 Hz), 7.27 (s, 1H), 7.25 (s, 1H), 7.10-7.20 (m, 5H), 6.83 (d, 1H, J=2.8 Hz), 6.71-6.74 (m, 4H), 6.49 (t, 1H, J=5.6 Hz), 4.89-4.93 (m, 1H), 4.11 (d, 1H, J=4.4 Hz), 3.73-3.77 (m, 2H), 3.702 (s, 3H), 3.698 (s, 3H), 3.56-3.66 (m, 1H), 3.37-3.45 (m, 4H), 3.17 (s, 3H), 3.08 (s, 6H), 2.95 (s, 3H), 2.38 (ddd, 1H, J=4.4, 7.2, 13.6 Hz); MS (FAB, NBA/CHCl₃) m/z 834 [(M+H)⁺]; HRMS (FAB) calculated for C₄₉H₅₁O₆N₇ [(M+H)⁺] 833.3901, found 834.3984.

### Synthesis of Compound (24) ((dimethylaminomethylidene)amino-8-DAN-2′-deoxy-3′-O-(cyanoethyl-N,N-diisopropylphosphoramidite)-5′-O-(4,4′-dimethoxytrityl)adenine)

To a solution of compound (23) (37 mg, 0.04 mmol) and tetrazole (3.1 mg, 0.044 mmol, 1.1 eq.) in anhydrous acetonitrile (0.4 ml, for synthesis of nucleic acid) was added 2-cyanoethyl tetraisopropylphosphorodiamidite (15 μl, 0.048 mmol, 1.2 eq.) under nitrogen. The mixture was stirred at room temperature for 30 min. The mixture was filtered and used with no further purification. Compound (24) is present in this mixture.

### Example 9

### Synthesis of an Oligonucleotide Derivative

Using compound (24) (PRODAN dA amidite) obtained in Example 8, oligodeoxyribonucleotide derivative 5′-CGCAACnCAACGC-3′ (SEQ ID No. 18) (wherein n is a PRODAN dA-containing deoxyribonucleotide, corresponding to compound (24)) was synthesized by conventional phosphoramidite method with an Applied Biosystems 392 DNA/RNA synthesizer.

The obtained oligodeoxyribonucleotide derivative was purified by reverse phase HPLC using a 5-ODS-H column (10×150 mm). Elution was performed with a linear gradient of 5% to 30% acetonitrile (0-30 min) and 30% to 40% acetonitrile (30-40 min) in 0.1 M triethylamine acetate (TEAA) buffer (pH 7.0) at a flow rate of 3.0 ml/min.

### Example 10

### Identification of an Oligonucleotide Derivative

Oligonucleotide derivatives containing PRODAN dA were fully digested with calf intestine alkaline phosphatase (50 U/ml), snake venom phosphodiesterase (0.15 U/ml) and P1 nuclease (50 U/ml) at 37° C. for 10 h. The digested solution was analyzed by HPLC on a 5-ODS-H column (4.6×150 mm) (elution with a solvent mixture of 0.1 M triethylammonium acetate (TEAA) buffer, pH 7.0, linear gradient 0-20% acetonitrile (0-20 min), 20-100% acetonitrile (20-30 min) at a flow rate 1.0 ml/min). The concentration of each oligonucleotide was determined by comparing peak areas with standard solution containing dA, dC, dG, and dT at a concentration of 0.1 mM.

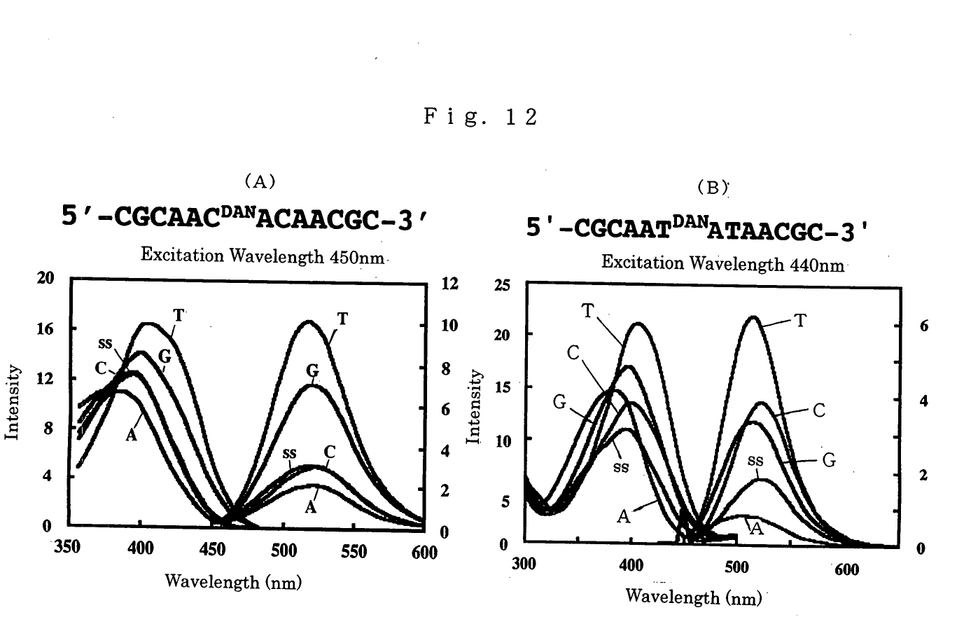

The HPLC elution pattern is shown in FIG. 11. In FIG. 11, X shows the elution peak of the sample obtained by the enzymatic degradation of the PRODAN dA-containing oligonucleotide drivative. A indicates deoxyadenylic acid (dA); C indicates deoxycytidylic acid (dC); G indicates deoxyguanylic acid (dG); and O indicates the uncleaved PRODAN dA-containing oligonucleotide derivative, which was not treated with the above-mentioned digestive enzymes. It is clear from FIG. 11 that the desired oligonucleotide drivative was synthesized.

### Example 11

### Fluorescence Analysis

The oligodeoxyribonucleotide derivative obtained in Example 9 was dissolved to a final concentration of 2.5 μM in a 50 mM phosphate buffer (pH 7.0) containing 0.1 M sodium chloride to prepare a solution. The fluorescence spectrum of the solution was measured at about 25° C. using a spectrophotofluorometer (RF5300PC, product of Shimadzu Corporation). The measurement was carried out at excitation wavelengths of 440 nm and 450 nm and an emission wavelength of 514 nm, with the excitation and emission band widths being 1.5 nm.

The fluorescence intensity of the single-strand oligonucleotide derivative was 3.1 at an excitation wavelength of 440 nm, and 1.8 at an excitation wavelength of 450 nm.

To samples of the above solution was added oligodeoxyribonucleotides (A′): 5′-GCGTTGAGTTGCG-3′ (SEQ ID No. 19), (T′): 5′-GCGTTGTGTTGCG-3′ (SEQ ID No. 20), (G′): 5′-GCGTTGGGTTGCG-3′ (SEQ ID No. 21), or (C′): 5′-GCGTTGCGTTGCG-3′ (SEQ ID No. 22), which are complementary to the oligodeoxyribonucleotide derivative (SEQ ID No. 18) in the portions other than nucleotide derivative (n), to a final concentration of 2.5 μM. Each solution sample was then mixed with a vortex mixer.

The fluorescence spectra of the solution samples were measured using the spectrophotofluorometer at the excitation wavelength of 440 nm. When oligodeoxyribonucleotide (A′) was added, the fluorescence intensity at 514 nm was 2.0. When oligodeoxyribonucleotide (T′) was added, the fluorescence intensity at 514 nm was 10.1. When oligodeoxyribonucleotide (G′) was added, the fluorescence intensity at 514 nm was 7.0. When oligodeoxyribonucleotide (C′) was added, the fluorescence intensity at 514 nm was 3.1.

The fluorescence spectra of the solution samples were measured using the spectrophotofluorometer at the excitation wavelength of 450 nm. When oligodeoxyribonucleotide (A′) was added, the fluorescence intensity at 514 nm was 0.8. When oligodeoxyribonucleotide (T′) was added, the fluorescence intensity at 514 nm was 6.2. When oligodeoxyribonucleotide (G′) was added, the fluorescence intensity at 514 nm was 3.3. When oligodeoxyribonucleotide (C′) was added, the fluorescence intensity at 514 nm was 3.7.

As is clear from the above, when the nucleotide on the complementary strand that paired with the nucleotide derivative (n) of the oligodeoxyribonucleotide derivative (SEQ ID No. 18) was deoxythymidylic acid, the fluorescence intensity was increased to about 3.4 times the original intensity by hybridization. This degree of increase is much higher than in the cases of the other nucleotides.

The fluorescence spectra are shown in FIG. 12. FIG. 12 (A) shows the results obtained at the excitation wavelength of 440 nm, and FIG. 12(B) the results obtained at the excitation wavelength of 450 nm. In FIG. 12, the vertical axis scale on the left shows absorbance for the absorption spectra, and the vertical axis scale on the right shows the fluorescence intensity of the fluorescence spectra.

### Example 12

When oligonucleotide derivatives containing one or more nucleotide derivatives represented by formulae (21), (23) or (24) are used, the resulting patterns are similar to those of Example 4 and Example 11.

1. A compound represented by formula (1): 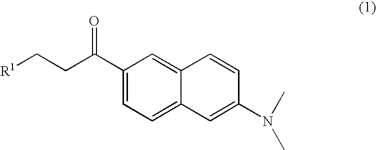
wherein R¹ is a substituent represented by formula (2): 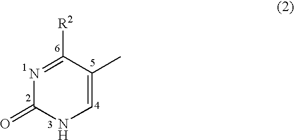
wherein R² is ═O or —NH₂, with the proviso that when R² is ═O, H is attached to the 1-position N of the pyrimidine ring, and the bond between the 1-position N and the 6-position C is a single bond; or a substituent represented by formula (3): 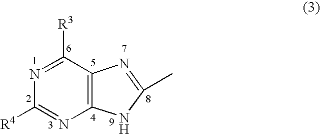
wherein R³ is —OH, ═O, or —NH₂, with the proviso that when R³ is —OH or —NH₂, R⁴ is H; when R³ is ═O, R⁴ is —NH₂; and when R³ is ═O, H is attached to the 1-position N of the purine ring, and the bond between the 1-position N and the 6-position C is a single bond.

2. The compound according to claim 1 represented by formula (4):

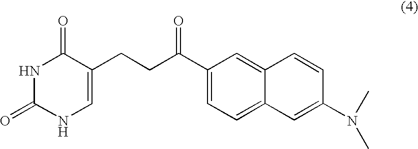

3. The compound according to claim 1 represented by formula (5):

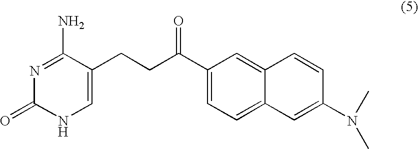

4. The compound according to claim 1 represented by formula (6):

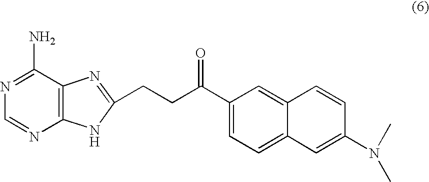

5. The compound according to claim 1 represented by formula (7):

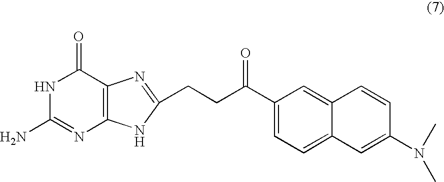

6. The compound according to claim 1 represented by formula (8):

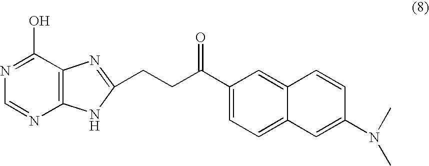

7. A nucleoside or nucleotide derivative represented by formula (9): 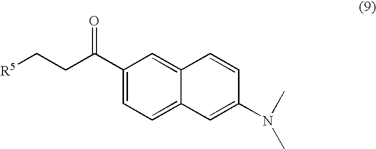
wherein R⁵ is a substituent represented by formula (10): 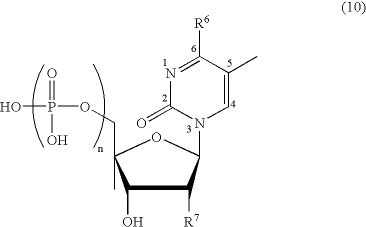
wherein R⁷ is —H or —OH; n is 0, 1, 2, or 3; and R⁶ is ═O or —NH₂, with the proviso that when R⁶ is ═O, H is attached to the 1-position N of the pyrimidine ring, and the bond between the 1-position N and the 6-position C is a single bond; or a substituent represented by formula (11): 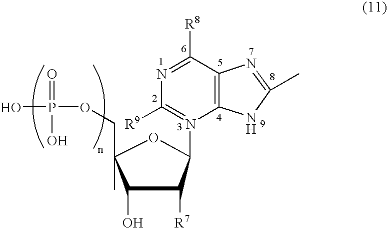
wherein R⁷ is —H or —OH; n is 0, 1, 2, or 3; and R⁸ is —OH, ═O, or —NH₂, with the proviso that when R⁸ is —OH or —NH₂, R⁹ is H; when R⁸ is ═O, R⁹ is —NH₂; and when R⁸ is ═O, H is attached to the 1-position N of the purine ring, and the bond between the 1-position N and the 6-position C is a single bond.

8. A nucleoside or nucleotide derivative according to claim 7 represented by formula (12): 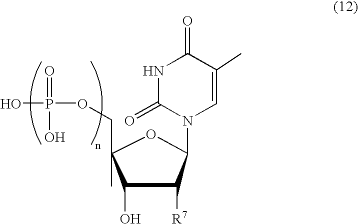
wherein R⁷ is —H or —OH; and n is 0, 1, 2, or 3.

9. A nucleoside or nucleotide derivative according to claim 7 represented by formula (13): 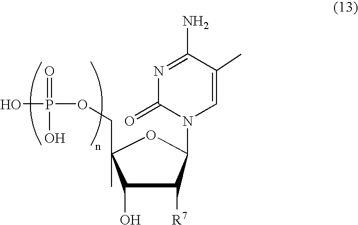
wherein R⁷ is —H or —OH; and n is 0, 1, 2, or 3.

10. A nucleoside or nucleotide derivative according to claim 7 represented by formula (14): 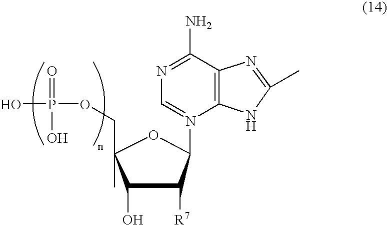
wherein R⁷ is —H or —OH; and n is 0, 1, 2, or 3.

11. A nucleoside or nucleotide derivative according to claim 7 represented by formula (15): 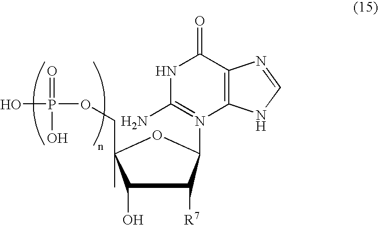
wherein R⁷ is —H or —OH; and n is 0, 1, 2, or 3.

12. A nucleoside or nucleotide derivative according to claim 7 represented by formula (16): 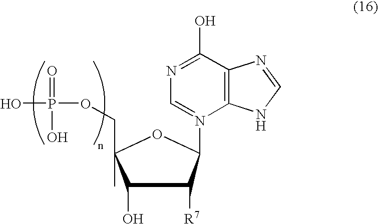
wherein R⁷ is —H or —OH; and n is 0, 1, 2, or 3.

13. A polynucleotide derivative wherein one or more nucleotides are substituted by a nucleotide derivative represented by formula (17): 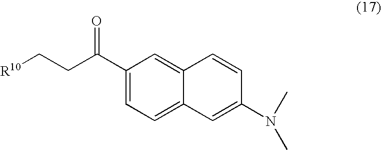
wherein R¹⁰ is a substituent represented by formula (18): 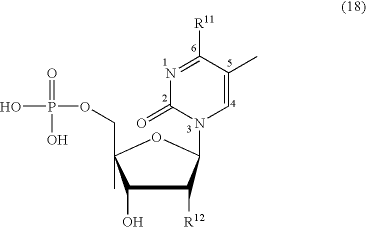
wherein R¹² is —H or —OH; and R¹¹ is ═O or —NH₂, with the proviso that when R¹¹ is ═O, H is attached to the 1-position N of the pyrimidine ring, and the bond between the 1-position N and the 6-position C is a single bond; or a substituent represented by formula (19): 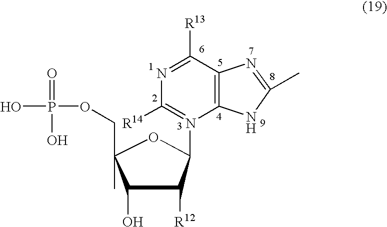
wherein R¹² is —H or —OH; and R¹³ is —OH, ═O, or —NH₂, with the proviso that when R¹³ is —OH or —NH₂, R¹⁴ is H; when R¹³ is ═O, R¹⁴ is —NH₂; and when R¹³ is ═O, H is attached to the 1-position N of the purine ring, and the bond between the 1-position N and the 6-position C is a single bond.

14. A polynucleotide derivative according to claim 13 wherein one or more nucleotides are substituted by a nucleotide derivative represented by formula (20): 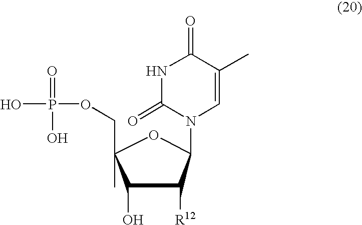
wherein R¹² is —H or —OH.

15. A polynucleotide derivative according to claim 13 wherein one or more nucleotides are substituted by a nucleotide derivative represented by formula (21): 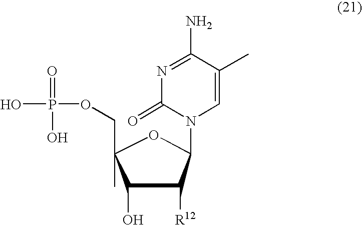
wherein R¹² is —H or —OH.

16. A polynucleotide derivative according to claim 13 wherein one or more nucleotides are substituted by a nucleotide derivative represented by formula (22): 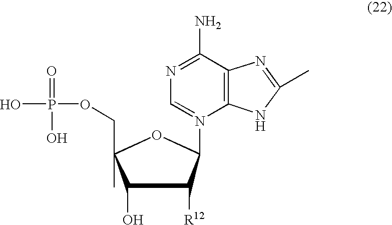
wherein R¹² is —H or —OH.

17. A polynucleotide derivative according to claim 13 wherein one or more nucleotides are substituted by a nucleotide derivative represented by formula (23): 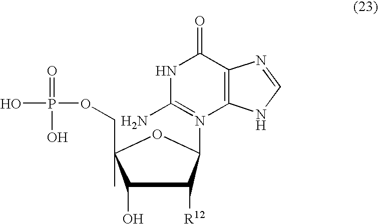
wherein R¹² is —H or —OH.

18. A polynucleotide derivative according to claim 13 wherein one or more nucleotides are substituted by a nucleotide derivative represented by formula (24): 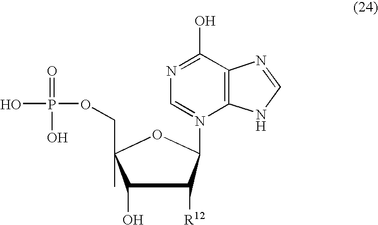
wherein R¹² is —H or —OH.

19. A compound represented by formula (25): 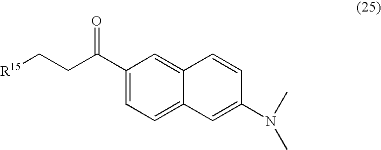
wherein R¹⁵ is a substituent represented by formula (26): 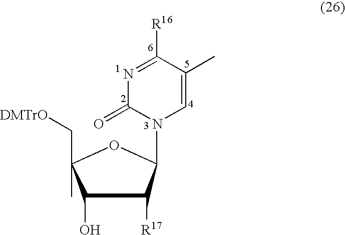
wherein R¹⁷ is —H or —OH; DMTrO— is a dimethoxytrityl group; and R¹⁶ is ═O or —NH₂, with the proviso that when R¹⁶ is ═O, H is attached to the 1-position N of the pyrimidine ring, and the bond between the 1-position N and the 6-position C is a single bond; or a substituent represented by formula (27): 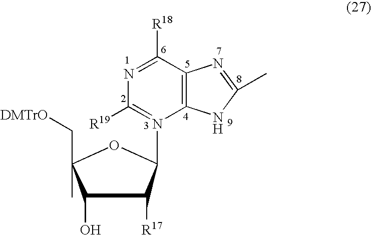
wherein R¹⁷ is —H or —OH; DMTrO— is a dimethoxytrityl group; and R¹⁸ is —OH, ═O, or —NH₂, with the proviso that when R¹⁸ is —OH or —NH₂, R¹⁹ is H; when R¹⁸ is ═O, R¹⁹ is —NH₂; and when R¹⁸ is ═O, H is attached to the 1-position N of the purine ring, and the bond between the 1-position N and the 6-position C is a single bond.

20. A compound according to claim 19 represented by formula (28): 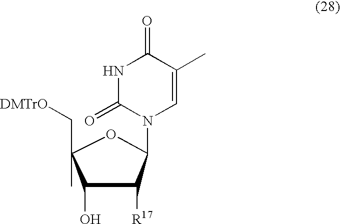
wherein R¹⁷ is —H or —OH; and DMTrO— is a dimethoxytrityl group.

21. A compound according to claim 19 represented by formula (29): 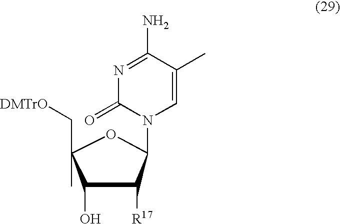
wherein R¹⁷ is —H or —OH; and DMTrO— is a dimethoxytrityl group.

22. A compound according to claim 19 represented by formula (30): 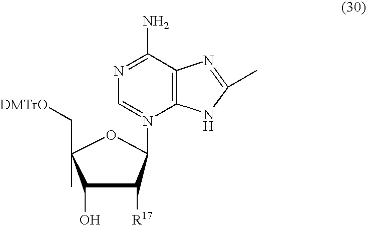
wherein R¹⁷ is —H or —OH; and DMTrO— is a dimethoxytrityl group.

23. A compound according to claim 19 represented by formula (31): 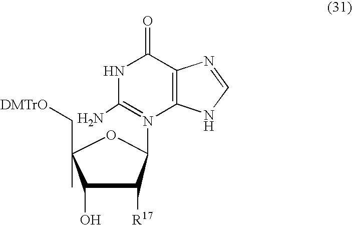
wherein R ¹⁷ is —H or —OH; and DMTrO— is a dimethoxytrityl group.

24. A compound according to claim 19 represented by formula (32): 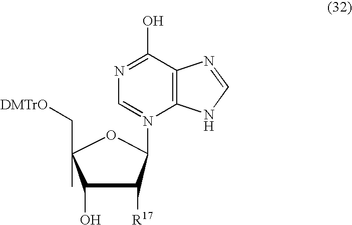
wherein R¹⁷ is —H or —OH; and DMTrO— is a dimethoxytrityl group.

25. A compound represented by formula (33): 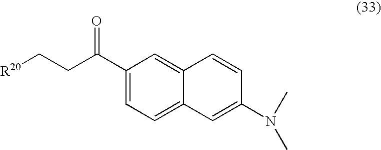
wherein R²⁰ is a substituent represented by formula (34): 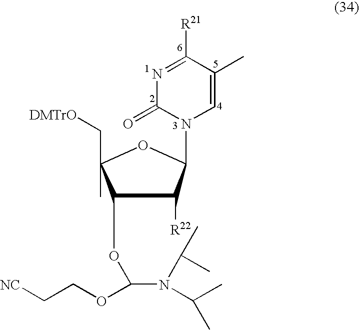
wherein R²² is —H or —OH; DMTrO— is a dimethoxytrityl group; and R²¹ is ═O or —NH₂, with the proviso that when R²¹ is ═O, H is attached to the 1-position N of the pyrimidine ring, and the bond between the 1-position N and the 6-position C is a single bond; or a substituent represented by formula (35): 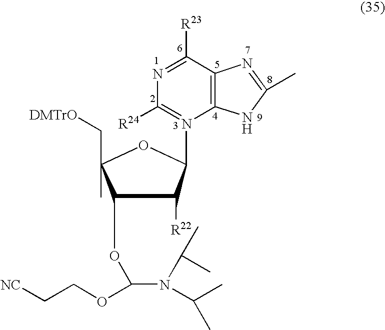
wherein R²² is —H or —OH; DMTrO— is a dimethoxytrityl group; and R²³ is —OH, ═O, or —NH₂, with the proviso that when R²³ is —OH or —NH₂, R²⁴ is H; when R²³ is ═O, R²⁴ is —NH₂; and when R²³ is ═O, H is attached to the 1-position N of the purine ring, and the bond between the 1-position N and the 6-position C is a single bond.

26. A compound according to claim 25 represented by formula (36): 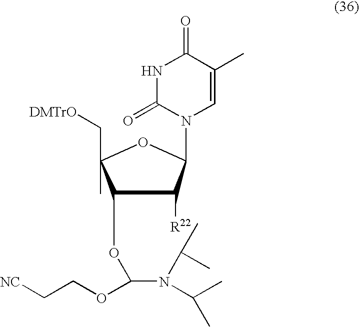
wherein R¹⁷ is —H or —OH; and DMTrO— is a dimethoxytrityl group.

27. A compound according to claim 25 represented by formula (37): 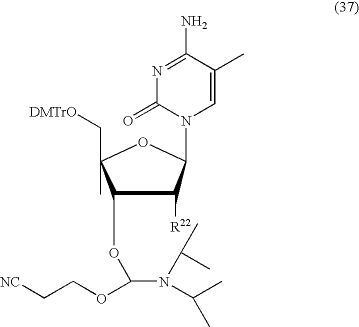
wherein R17 is —H or —OH; and DMTRO— is a dimethoxytrityl group.

28. A compound according to claim 25 represented by formula (38): 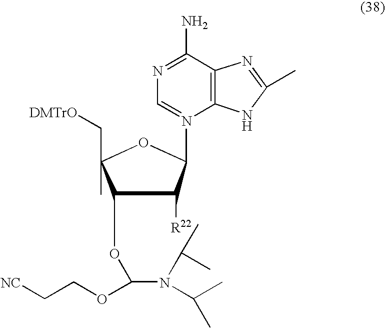
wherein R¹⁷ is —H or —OH; and DMTrO— is a dimethoxytrityl group.

29. A compound according to claim 25 represented by formula (39): 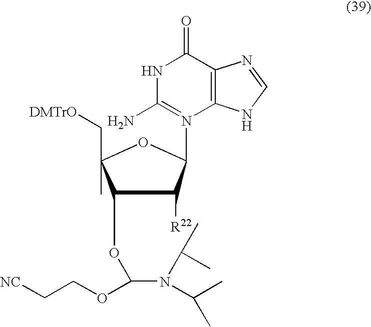
wherein R¹⁷ is —H or —OH; and DMTrO— is a dimethoxytrityl group.

30. A compound according to claim 25 represented by formula (40): 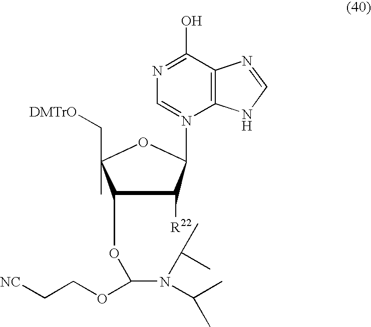
wherein R¹⁷ is —H or —OH; and DMTrO— is a dimethoxytrityl group.

31. A probe comprising a polynucleotide derivative according to any one of claims 13 to 18.

32. A nucleotide identification reagent comprising a probe according to claim 31.

33. A nucleic acid quantitation reagent comprising a probe according to claim 31.

34. A DNA chip wherein one or more polynucleotide derivatives according to any one of claims 13 to 18 are immobilized or adsorbed onto a substrate.

35. A method of identifying a nucleotide in a target nucleic acid, comprising the steps of:
hybridizing a polynucleotide derivative according to any one of claims 13 to 18 with a target nucleic acid in a sample;
measuring a fluorescence spectrum of the hybridization product; and
comparing the fluorescence spectrum of the hybridization product with a fluorescence spectrum of the polynucleotide derivative before hybridization, thereby identifying a nucleotide at a particular position in the target nucleic acid.

36. A primer comprising a polynucleotide derivative according to any one of claims 13 to 18.

37. A nucleic acid quantitation reagent comprising a primer according to claim 36.

38. A method of quantitating a target nucleic acid in a sample, comprising the steps of:
hybridizing a polynucleotide derivative according to any one of claims 13 to 18 with a target nucleic acid in a sample;
measuring the fluorescence intensity of the hybridization product; and
comparing the fluorescence intensity of the hybridization product with the fluorescence intensity of a hybridization product obtained by hybridizing the above polynucleotide derivative with a target nucleic acid whose concentration is known, thereby determining the concentration of the target nucleic acid.
